(19) United States
(12) Patent Application Publication
(10) Pub. No.: US 2024/0368068 A1
(43) Pub. Date: Nov. 7, 2024

(54) PROCESS FOR PREPARING PERFUMING INTERMEDIATES

(71) Applicant: FIRMENICH SA, Satigny (CH)

(72) Inventors: Oliver KNOPFF, Satigny (CH); Nicolas POIRIER, Satigny (CH)

(21) Appl. No.: 18/689,631

(22) PCT Filed: Sep. 5, 2022
(86) PCT No.: PCT/EP2022/074600

§ 371 Date: Mar. 6, 2024

(30) Foreign Application Priority Data
Sep. 7, 2021 (EP) 21195368.2

(51) Int. Cl. C07C 67/475; C07C 29/147; C07C 33/02; C07C 41/18; C07C 43/178; C07C 45/29; C07C 45/54; C07C 47/42; C07C 69/757

(52) U.S. Cl. CPC C07C 67/475; C07C 29/147; C07C 33/02; C07C 41/18; C07C 43/1785; C07C 45/29; C07C 45/54; C07C 47/42; C07C 69/757; C07C 2601/14

(57) ABSTRACT

The present invention relates to the field of perfumery. More particularly, it concerns valuable new chemical intermediates for producing perfuming ingredients. Moreover, the present invention comprises also a process for producing compound of formula (I).

### TECHNICAL FIELD

The present invention relates to the field of perfumery. More particularly, it concerns valuable new chemical intermediates for producing perfuming ingredients. Moreover, the present invention comprises also a process for producing compound of formula (I).

### BACKGROUND OF THE INVENTION

In the perfumery industry, there is a constant need to provide compounds imparting novel organoleptic notes. In particular, there is an interest towards aldehydic notes which represents one of the key organoleptic facets of the lily of the valley odor. So, compounds imparting said note are particularly sought after to reconstitute the delicate floral odor of muguet which does not survive even the mildest of extraction methods to yield an essential oil. 3-(cyclohex-1-en-1-yl) propanal, 3-phenyl propanal or 3-phenyl pentenal derivatives represent compounds imparting note of the muguet-aldehydic olfactive family, such as, for example, 3-(4,4-dimethyl-1-cyclohexen-1-yl)propanal reported in EP 1529770, 3-[4-(2-methyl-2-propanyl)-1-cyclohexen-1-yl]propanal reported in EP 1054053 or 4-methyl-5-(4-methylphenyl)-4-pentenal reported in WO 2010052635. However, the access to these derivatives is tedious and requires Grignard reagents, radical chemistry or pyrolysis providing the desired compounds with low yield and/or selectivity.

Being products of industrial interest, there is always a need for new processes showing an improved yield or productivity.

The present invention allows obtaining compound of formula (I), by cross metathesis between commercially available or easily available compounds of formula (II) and (III) while avoiding a use of toxic acrolein or crotonaldehyde, which may be easily converted into compound of formula (V). The compounds of formula (I), (IV) and (VII), key intermediates in this process, have never been reported or suggested in the context of the preparation of compounds of formula (V).

### SUMMARY OF THE INVENTION

The invention relates to a novel process allowing the preparation of novel compound of formula (I) offering a new access to compound of formula (V) with high yield and selectivity. The invention process represents a new efficient route toward compound of formula (V).

So, the first object of the present invention is a process for the preparation of a compound of formula

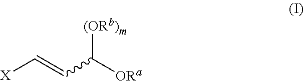

in the form of any one of its stereoisomers or a mixture thereof, and wherein m is 0 or 1; R^{a} is a C₁₋₄ alkyl group, a trimethyl silyl group, a C(═O)—R^{c} group, a C(═O)—OR^{c} group, a CH₂(OR^{c}) group, a CH(OR^{c})CH₃ group wherein R^{c} is a C₁₋₄ alkyl group; R^{b} is a C₁₋₄ alkyl group, a C(═O)—R^{c} group wherein R^{c} is a C₁₋₄ alkyl group; or R^{a} and R^{b} are taken together and represent a C₂₋₆ alkanediyl group X is a group of formula a), b) or c)

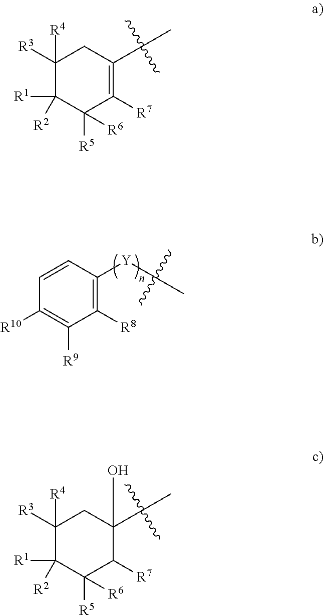

in the form of any one of its stereoisomers, and wherein each R¹, R², R³, R⁴, R⁵, R⁶ and R⁷, independently from each other, represent a hydrogen atom, a C₁₋₆ alkyl group or a C₂₋₆ alkenyl group, each optionally substituted by a hydroxy or C₁₋₃ alkoxy group; or two groups among R¹, R², R³, R⁴, R⁵, R⁶ and R⁷ are taken together and form C₃₋₈ cycloalkyl or C₅₋₈ cycloalkenyl group, each optionally substituted by a hydroxy, C₁_3 alkyl or C₁₋₃ alkoxy group, and the others groups have the same meaning as defined above; each R⁸, R⁹ and R¹⁰, independently from each other, represent a hydrogen atom, a C₁₋₆ alkyl group or a C₂₋₆ alkenyl group, each optionally substituted by a hydroxy or C₁₋₃ alkoxy group; or two groups among R⁸, R⁹ and R¹⁰ are taken together and form C₃₋₈ cycloalkyl or C₅₋₈ cycloalkenyl group, each optionally substituted by a hydroxy, C₁₋₃ alkyl or C₁₋₃ alkoxy group, and the other group has the same meaning as defined above; n is 0 or 1 and Y is a —CH═CR¹¹—, —CHR¹²—CHR¹¹- or —CH₂—C(OH)R¹¹— group wherein R¹¹ is a hydrogen atom or a methyl or ethyl group and R¹² is a hydroxy or a acetate group; provided that m is 1 when X is a group of formula b); comprising a cross metathesis step, in the presence of a metathesis catalyst, between compound of the formula (IIa), (IIb) or (IIc)

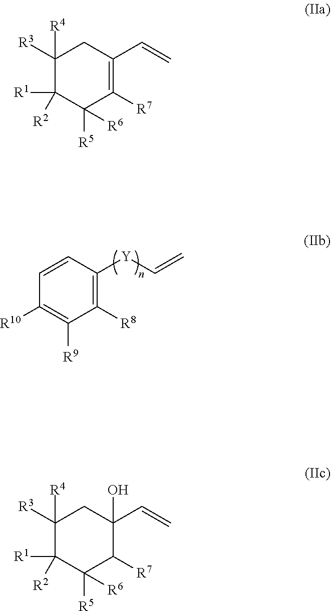

in the form of any one of its stereoisomers, and wherein each R¹, R², R³, R⁴, R⁵, R⁶, R⁷, R⁸, R⁹, R¹⁰, n and Y have the same meaning as defined above
with compound of formula (III)

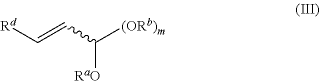

in the form of any one of its stereoisomers or a mixture thereof, and wherein m is 0 or 1 when the cross metathesis step is performed with compound of the formula (IIa) or (IIc) or m is 1 when the cross metathesis step is performed with compound of the formula (IIb), R^{a} is a C₁₋₄ alkyl group, a trimethyl silyl group, a C(═O)—R^{c} group, a C(═O)—OR^{c} group, a CH₂(OR^{c}) group, a CH(OR^{c})CH₃ group wherein R^{c} is a C₁₋₄ alkyl group; R^{b} is a C₁₋₄ alkyl group, a C(═O)—R^{c} group wherein R^{c} is a C₁₋₄ alkyl group; or R^{a} and R^{b} are taken together and represent a C₂₋₆ alkanediyl group; R^{d} is a hydrogen atom or a CH(OR^{a})(OR^{b})ₘ group wherein m, R^{a} and R^{b} have the same meaning as defined above.

Another object of the present invention is compound of formula

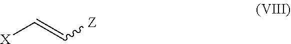

in the form of any one of its stereoisomers or a mixture thereof, and wherein X is a group of formula b) or d)

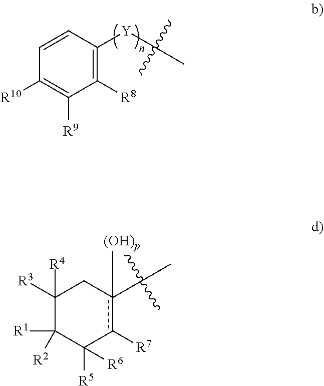

in the form of any one of its stereoisomers, and
wherein p is 0 when the dotted line is a carbon-carbon double bond and p is 1 when the dotted line is a carbon-carbon single bond;
each R¹, R², R³, R⁴, R⁵, R⁶ and R⁷, independently from each other, represent a hydrogen atom, a C₁₋₆ alkyl group or a C₂-6 alkenyl group, each optionally substituted by a hydroxy or C₁₋₃ alkoxy group; or two groups among R¹, R², R³, R⁴, R⁵, R⁶ and R⁷ are taken together and form C₃₋₈ cycloalkyl or C₅₋₈ cycloalkenyl group, each optionally substituted by a hydroxy, C₁₋₃ alkyl or C₁₋₃ alkoxy group, and the others groups have the same meaning as defined above;
each R⁸, R⁹ and R¹⁰, independently from each other, represent a hydrogen atom, a C₁₋₆ alkyl group or a C₂₋₆ alkenyl group, each optionally substituted by a hydroxy or C₁₋₃ alkoxy group; or two groups among R⁸, R⁹ and R¹⁰ are taken together and form C₃₋₈ cycloalkyl or C₅₋₈ cycloalkenyl group, each optionally substituted by a hydroxy, C₁_3 alkyl or C₁₋₃ alkoxy group, and the other group has the same meaning as defined above;
n is 0 or 1;
Y is a —CH═CR¹¹—, —CHR¹²—CHR¹¹- or —CH₂—C(OH)R¹¹— group wherein R¹¹ is a hydrogen atom or a methyl or ethyl group and R¹² is a hydroxy or a acetate group;
Z is, when X is of formula d), a CHO group, a CH₂OH group or a CH(OR^{a})(OR^{b})ₘ group wherein m is 0 or 1; R^{a} is a C₁₋₄ alkyl group, a trimethyl silyl group, a C(═O)—R^{c} group, a C(═O)—OR^{c} group, a CH₂(OR^{c}) group, a CH(OR^{c})CH₃ group wherein R^{c} is a C₁₋₄ alkyl group; R^{b} is a C₁₋₄ alkyl group, a C(═O)—R^{c} group wherein R^{c} is a C₁₋₄ alkyl group; or R^{a} and R^{b} are taken together and represent a C₂₋₆ alkanediyl group;
Z is, when X is of formula b), a CH(OR^{a})(OR^{b}) group wherein R^{a} and R^{b}, independently from each other, are a methyl group or a C₃₋₄ alkyl group, a C(═O)—R^{c} group wherein R^{c} is a C₁₋₄ alkyl group; or R^{a} and R^{b} are taken together and represent a C₃₋₆ alkanediyl group; and
provided that when X is a group of formula (d), then at least one group among R¹, R², R³, R⁴, R⁵, R⁶ and R⁷ is a C₁₋₆ alkyl group or a C₂₋₆ alkenyl group, each optionally substituted by a hydroxy or C₁₋₃ alkoxy group; provided that when X is a group of formula (d) and six groups among R¹, R², R³, R⁴, R⁵, R⁶ and R⁷ are a hydrogen atom, then the group among R¹, R², R³, R⁴, R⁵, R⁶ and R⁷ being not a hydrogen atom is not a methyl group; provided that when X is a group of formula (d) and R², R³, R⁴, R⁵, R⁶ and R⁷ are a hydrogen atom then R¹ is not a prop-1-en-2-yl group; and provided that 3-(4-(tert-butyl)cyclohex-1-en-1-yl)acrylaldehyde, 3-(4-(tert-butyl)-1-hydroxycyclohexyl)acrylaldehyde, 1-(tert-butyl)-4-(3,3-diethoxyprop-1-en-1-yl)benzene, 2-(4-(tert-butyl)styryl)-1,3-dioxolane, 3-(4-(tert-butyl)-1-hydroxycyclohexyl)allyl acetate, 3-(2-allyl-1-hydroxycyclohexyl)allyl acetate, 4-(tert-butyl)-1-(3-hydroxyprop-1-en-1-yl)cyclohexan-1-ol, 2-allyl-1-(3-hydroxyprop-1-en-1-yl)cyclohexan-1-ol, 2-(4-isopropylstyryl)-1,3-dioxane, 2-(4-isopropylstyryl)-4-methyl-1,3-dioxolane, 3-(2-(buta-1,3-dien-1-yl)-1-hydroxycyclohexyl)acrylaldehyde, 3-(5,5-dimethylcyclohex-1-en-1-yl)acrylaldehyde, 3-((1S,5R)-6,6-dimethylbicyclo[3.1.1]hept-2-en-3-yl)acrylaldehyde, (1R,2S,5R)-1-(3-(tert-butoxy)prop-1-en-1-yl)-2-isopropyl-5-methylcyclohexan-1-ol, 2-allyl-1-(3-hydroxyprop-1-en-1-yl)cyclohexan-1-ol, (3,3-dimethoxyprop-1-en-1-yl)benzene, are excluded.

### DESCRIPTION OF THE INVENTION

Surprisingly, it has now been discovered that compound of formula (I), key building block toward perfuming ingredients, can be produced in an advantageous manner by means of a cross metathesis reaction between compound of formula (II) and compound of formula (III). The invention's conditions allow a straightforward access to valuable perfuming ingredients 3-(cyclohex-1-en-1-yl) propanal derivatives, 3-(phenyl) propanal derivatives or 5-(phenyl)-4-pentenal of formula (V) using novel intermediates never reported in the art such as compound of formula (I) and (IV).

So, the first object of the invention is a process for the preparation of a compound of formula

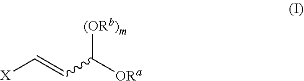

in the form of any one of its stereoisomers or a mixture thereof, and wherein m is 0 or 1; R^{a} is a C₁₋₄ alkyl group, a trimethyl silyl group, a C(═O)—R^{c} group, a C(═O)—OR^{c} group, a CH₂(OR^{c}) group, a CH(OR^{c})CH₃ group wherein R^{c} is a C₁₋₄ alkyl group; R^{b} is a C₁₋₄ alkyl group, a C(═O)—R^{c} group wherein R^{c} is a C₁₋₄ alkyl group; or R^{a} and R^{b} are taken together and represent a C₂₋₆ alkanediyl group X is a group of formula a), b) or c)

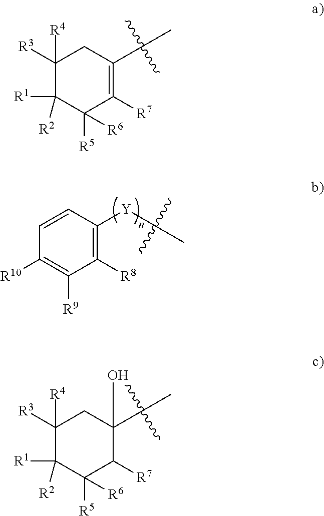

in the form of any one of its stereoisomers, and wherein each R¹, R², R³, R⁴, R⁵, R⁶ and R⁷, independently from each other, represent a hydrogen atom, a C₁₋₆ alkyl group or a C₂₋₆ alkenyl group, each optionally substituted by a hydroxy or C₁₋₃ alkoxy group; or
two groups among R¹, R², R³, R⁴, R⁵, R⁶ and R⁷ are taken together and form C₃₋₈ cycloalkyl or C₅₋₈ cycloalkenyl group, each optionally substituted by a hydroxy, C₁_3 alkyl or C₁₋₃ alkoxy group, and the others groups have the same meaning as defined above; each R⁸, R⁹ and R¹⁰, independently from each other, represent a hydrogen atom, a C₁₋₆ alkyl group or a C₂-6 alkenyl group, each optionally substituted by a hydroxy or C₁₋₃ alkoxy group; or two groups among R⁸, R⁹ and R¹⁰ are taken together and form C₃₋₈ cycloalkyl or C₅₋₈ cycloalkenyl group, each optionally substituted by a hydroxy, C₁₋₃ alkyl or C₁₋₃ alkoxy group, and the other group has the same meaning as defined above; n is 0 or 1 and Y is a —CH═CR¹¹—, —CHR¹²—CHR¹¹— or —CH₂—C(OH)R¹¹— group wherein R¹¹ is a hydrogen atom or a methyl or ethyl group and R¹² is a hydroxy or a acetate group; provided that m is 1 when X is a group of formula b);
   comprising a cross metathesis step, in the presence of a metathesis catalyst, between compound of the formula (IIa), (IIb) or (IIc)

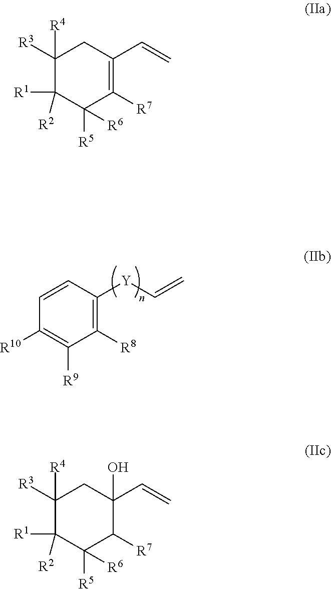

in the form of any one of its stereoisomers, and wherein each R¹, R², R³, R⁴, R⁵, R⁶, R⁷, R⁸, R⁹, R¹⁰, n and Y have the same meaning as defined above
with compound of formula (III)

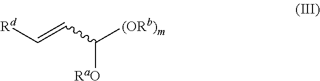

in the form of any one of its stereoisomers or a mixture thereof, and wherein m is 0 or 1 when the cross metathesis step is performed with compound of the formula (IIa) or (IIc) or m is 1 when the cross metathesis step is performed with compound of the formula (IIb), R^{a} is a C₁₋₄ alkyl group, a trimethyl silyl group, a C(═O)—R^{c} group, a C(═O)—OR^{c} group, a CH₂(OR^{c}) group, a CH(OR^{c})CH₃ group wherein R^{c} is a C₁₋₄ alkyl group; R^{b} is a C₁₋₄ alkyl group, a C(═O)—R^{c} group wherein R^{c} is a C₁₋₄ alkyl group; or R^{a} and R^{b} are taken together and represent a C₂₋₆ alkanediyl group, R^{d} is a hydrogen atom or a CH(OR^{a})(OR^{b})ₘ group wherein m, R^{a} and R^{b} have the same meaning as defined above.

For the sake of clarity, by the wavy bond in compounds of formula (I) and (III), or the similar, it is meant the normal meaning understood by a person skilled in the art, i.e. that the double bond may have a cis configuration, a trans configuration or a mixture thereof.

For the sake of clarity, by the expression “any one of its stereoisomers or a mixture thereof”, or the similar, it is meant the normal meaning understood by a person skilled in the art, i.e. that the compound of formula (I), (II) and (III) can be a pure enantiomer or a mixture of enantiomers. In other words, the compound of formula (I), (II) and (III) may possess at least one stereocenter which can have two different stereochemistries (e.g. R or S). The compounds of formula (I), (II) and (III) may even be in the form of a pure enantiomer or in the form of a mixture of enantiomers. The compounds of formula (I), (II) and (III) may even be in the form of a pure diastereoisomer or in the form of a mixture of diastereoisomer when compounds of formula (I), (II) and (III) possess more than one stereocenter. The compounds of formula (I), (II) and (III) can be in a racemic form or scalemic form. Therefore, the compounds of formula (I), (II) and (III) can be one stereoisomer or in the form of a composition of matter comprising, or consisting of, various stereoisomers. In addition, said compound of formula (I) or (III) can be in the form of its E or Z isomer or of a mixture thereof, e.g. the invention comprises compositions of matter consisting of one or more compounds of formula (I) or (III), having the same chemical structure but differing by the configuration of the double bond.

The term “optionally” is understood that a certain group to be optionally substituted can or cannot be substituted with a certain functional group.

The terms “alkyl” and “alkenyl” are understood as comprising branched and linear alkyl and alkenyl groups. The terms “alkenyl” and “cycloalkenyl” are understood as comprising 1, 2 or 3 olefinic double bonds, preferably 1 or 2 olefinic double bonds. In particular, for alkenyl, the olefinic double bound is not a terminal double bond. The terms “cycloalkyl” and “cycloalkenyl” are understood as comprising a monocyclic or fused, spiro and/or bridged bicyclic or tricyclic cycloalkyl and cycloalkenyl, groups, preferably monocyclic cycloalkyl and cycloalkenyl groups.

For the sake of clarity, by the expression “two groups among R¹, R², R³, R⁴, R⁵, R⁶ and R⁷ are taken together and form C₃₋₈ cycloalkyl or C₅₋₈ cycloalkenyl group”, it is meant that the carbon atom(s) to which both groups are bonded is/are included into the C₅₋₈ cycloalkyl or C₅₋₈ cycloalkenyl group.

According to any embodiment of the invention, when m is 1, R^{a} and R^{b} are a C₁₋₄ alkyl group, a C(═O)—R^{c} group wherein R^{c} is a C₁₋₄ alkyl group; or R^{a} and R^{b} are taken together and represent a C₂₋₆ alkanediyl group. In particular, when m is 1, R^{a} and R^{b} are identical.

According to any embodiment of the invention, the invention's process is a process for the preparation of a compound of formula

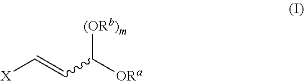

in the form of any one of its stereoisomers or a mixture thereof, and wherein m is 0 or 1; R^{a} is a C₁₋₄ alkyl group, a trimethyl silyl group, a C(═O)—R^{c} group, a C(═O)—OR^{c} group, a CH₂(OR^{c}) group, a CH(OR^{c})CH₃ group wherein R^{c} is a C₁₋₄ alkyl group; R^{b} is a C₁₋₄ alkyl group, a C(═O)—R^{c} group wherein R^{c} is a C₁₋₄ alkyl group; or R^{a} and R^{b} are taken together and represent a C₂₋₆ alkanediyl group; X is a group of formula a) or c)

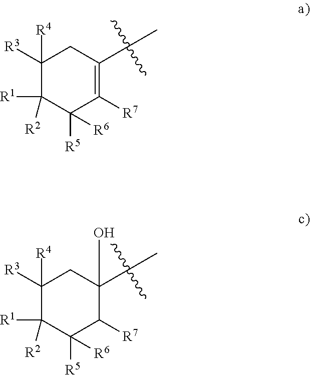

in the form of any one of its stereoisomers, and wherein each R¹, R², R³, R⁴, R⁵, R⁶ and R⁷, independently from each other, represent a hydrogen atom, a C₁₋₆ alkyl group or a C₂₋₆ alkenyl group, each optionally substituted by a hydroxy or C₁₋₃ alkoxy group; or two groups among R¹, R², R³, R⁴, R⁵, R⁶ and R⁷ are taken together and form C₃₋₈ cycloalkyl or C₅₋₈ cycloalkenyl group, each optionally substituted by a hydroxy, C₁₋₃ alkyl or C₁₋₃ alkoxy group, and the others groups have the same meaning as defined above;
   comprising a cross metathesis step, in the presence of a metathesis catalyst, between compound of the formula (IIa) or (IIc)

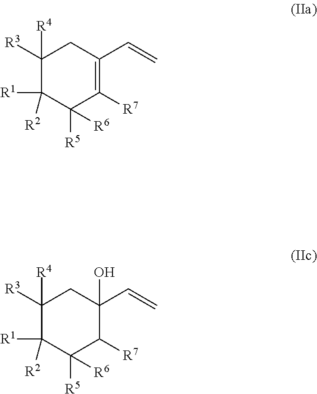

in the form of any one of its stereoisomers, and wherein each R¹, R², R³, R⁴, R⁵, R⁶ and R⁷ have the same meaning as defined above
with compound of formula (III)

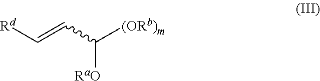

in the form of any one of its stereoisomers or a mixture thereof, and wherein m is 0 or 1; R^{a} is a C₁₋₄ alkyl group, a trimethyl silyl group, a C(═O)—R^{c} group, a C(═O)—OR^{c} group, a CH₂(OR^{c}) group, a CH(OR^{c})CH₃ group wherein R^{c} is a C₁₋₄ alkyl group; R^{b} is a C₁₋₄ alkyl group, a C(═O)—R^{c} group wherein R^{c} is a C₁₋₄ alkyl group or R^{a} and R^{b} are taken together and represent a C₂₋₆ alkanediyl group, R^{d} is a hydrogen atom or a CH(OR^{a})(OR^{b})ₘ group.

According to any embodiment of the invention, the invention's process is a process for the preparation of a compound of formula

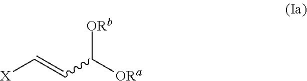

in the form of any one of its stereoisomers or a mixture thereof, and wherein R^{a} and R^{b}, independently from each other, are a C₁₋₄ alkyl group, a C(═O)—R^{c} group wherein R^{c} is a C₁₋₄ alkyl group; or R^{a} and R^{b} are taken together and represent a C₂-6 alkanediyl group; X is a group of formula b)

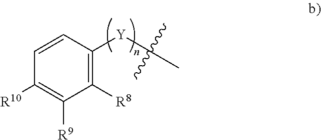

in the form of any one of its stereoisomers, and wherein each R⁸, R⁹ and R¹⁰, independently from each other, represent a hydrogen atom, a C₁₋₆ alkyl group or a C₂₋₆ alkenyl group, each optionally substituted by a hydroxy or C₁₋₃ alkoxy group; or two groups among R⁸, R⁹ and R¹⁰ are taken together and form C₃₋₈ cycloalkyl or C₅₋₈ cycloalkenyl group, each optionally substituted by a hydroxy, C₁₋₃ alkyl or C₁₋₃ alkoxy group, and the other group has the same meaning as defined above; n is 0 or 1 and Y is a —CH═CR¹¹—, —CHR¹²—CHR¹¹— or —CH₂—C(OH)R¹¹— group wherein R¹¹ is a hydrogen atom or a methyl or ethyl group and R¹² is a hydroxy or a acetate group;
   comprising a cross metathesis step, in the presence of a metathesis catalyst, between compound of the formula (IIb)

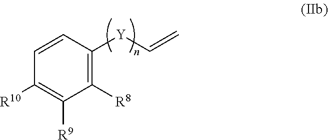

in the form of any one of its stereoisomers, and wherein R⁸, R⁹, R¹⁰, n and Y have the same meaning as defined above
with compound of formula (III)

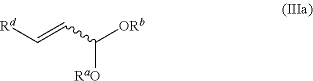

in the form of any one of its stereoisomers or a mixture thereof, and wherein R^{a} and R^{b}, independently from each other, are a C₁₋₄ alkyl group, a C(═O)—R^{c} group wherein R^{c} is a C₁₋₄ alkyl group; or R^{a} and R^{b} are taken together and represent a C₂₋₆ alkanediyl group, R^{d} is a hydrogen atom or a CH(OR^{a})(OR^{b}) group.

According to any embodiment of the invention, at least one group among R¹, R², R³, R⁴, R⁵, R⁶ and R⁷ may be a C₁₋₆ alkyl group or a C₂₋₆ alkenyl group, each optionally substituted by a hydroxy or C₁₋₃ alkoxy group, and the others may be, independently from each other, a hydrogen atom, a C₁₋₆ alkyl group or a C₂₋₆ alkenyl group, each optionally substituted by a hydroxy or C₁₋₃ alkoxy group. Particularly, at least three groups among R¹, R², R³, R⁴, R⁵, R⁶ and R⁷ may be a hydrogen atom, the others, may be, independently from each other, a hydrogen atom, a C₁₋₆ alkyl group or a C₂₋₆ alkenyl group, each optionally substituted by a hydroxy or C₁₋₃ alkoxy group. Particularly, four groups among R¹, R², R³, R⁴, R⁵, R⁶ and R⁷ may be a hydrogen atom, the others, may be, independently from each other, a hydrogen atom, a C₁₋₆ alkyl group or a C₂₋₆ alkenyl group, each optionally substituted by a hydroxy or C₁₋₃ alkoxy group. Particularly, one, two, three or four groups among R¹, R², R³, R⁴, R⁵, R⁶ and R⁷ may be a C₁₋₆ alkyl group or a C₂₋₆ alkenyl group, each optionally substituted by a hydroxy or C₁₋₃ alkoxy group, and the others may be a hydrogen atom. Even more particularly, one or two groups among R¹, R², R³, R⁴, R⁵, R⁶ and R⁷ may be a C₁₋₆ alkyl group or a C₂₋₆ alkenyl group, each optionally substituted by a hydroxy or C₁₋₃ alkoxy group, and the others may be a hydrogen atom.

According to any embodiment of the invention, R³, R⁴, R⁵, R⁶ and R⁷, independently from each other, may be a hydrogen atom or a C₁₋₄ alkyl group, optionally substituted by a hydroxy or C₁₋₃ alkoxy group. Particularly, R³, R⁴, R⁵, R⁶ and R⁷, independently from each other, may be a hydrogen atom or a C₁₋₃ alkyl group. Particularly, R³, R⁴, R⁵, R⁶ and R⁷, independently from each other, may be a hydrogen atom.

According to a particular embodiment of the invention, R¹, R², R³, R⁶ and R⁷, independently from each other, may be a hydrogen atom and R⁴, and R⁵ may be a hydrogen atom or a C₁₋₃ alkyl group. Particularly, R¹, R², R³, R⁶ and R⁷, independently from each other, may be a hydrogen atom and R⁴ may be a hydrogen atom and R⁵ may be a C₁₋₃ alkyl group or R⁴ may be a C₁₋₃ alkyl group and R⁵ may be a hydrogen atom.

According to any embodiment of the invention, at least one group among R⁸, R⁹ and R¹⁰ may be a C₁₋₆ alkyl group or a C₂₋₆ alkenyl group, each optionally substituted by a hydroxy or C₁₋₃ alkoxy group, and the others may be, independently from each other, a hydrogen atom, a C₁₋₆ alkyl group or a C₂₋₆ alkenyl group, each optionally substituted by a hydroxy or C₁₋₃ alkoxy group. Particularly, at least one groups among R⁸, R⁹ and R¹⁰ may be a hydrogen atom, the others, may be, independently from each other, a hydrogen atom, a C₁₋₆ alkyl group or a C₂₋₆ alkenyl group, each optionally substituted by a hydroxy or C₁₋₃ alkoxy group. Particularly, two groups among R⁸, R⁹ and R¹⁰ may be a hydrogen atom, the others, may be, independently from each other, a hydrogen atom, a C₁₋₆ alkyl group or a C₂₋₆ alkenyl group, each optionally substituted by a hydroxy or C₁₋₃ alkoxy group. Particularly, one or two groups among R⁸, R⁹ and R¹⁰ may be a C₁₋₆ alkyl group or a C₂₋₆ alkenyl group, each optionally substituted by a hydroxy or C₁₋₃ alkoxy group, and the others may be a hydrogen atom. Even more particularly, one group among R⁸, R⁹ and R¹⁰ may be a C₁₋₆ alkyl group or a C₂₋₆ alkenyl group, each optionally substituted by a hydroxy or C₁₋₃ alkoxy group, and the others may be a hydrogen atom.

According to any embodiment of the invention, R⁸ and R⁹, independently from each other, may be a hydrogen atom or a C₁₋₄ alkyl group, optionally substituted by a hydroxy or C₁₋₃ alkoxy group. Particularly, R⁸ and R⁹, independently from each other, may be a hydrogen atom or a C₁₋₃ alkyl group. Particularly, R⁸ and R⁹, independently from each other, may be a hydrogen atom.

According to any embodiment of the invention, R¹⁰ may be a hydrogen atom or a C₁₋₄ alkyl group, optionally substituted by a hydroxy or C₁₋₃ alkoxy group. Particularly, R¹⁰ may be a C₁₋₄ alkyl group, optionally substituted by a hydroxy or C₁₋₃ alkoxy group. Particularly, R¹⁰ may be a C₁₋₃ alkyl group.

According to any embodiment of the invention, Y may be a —CH═CR¹¹— group wherein R¹¹ is a hydrogen atom or a methyl or ethyl group.

According to any embodiment of the invention, R¹¹ may be a hydrogen atom or a methyl group.

According to any embodiment of the invention, X may be a group of formula a) or c). Particularly, X may be a group of formula a).

According to any embodiment of the invention, the compound formula (I) is of formula

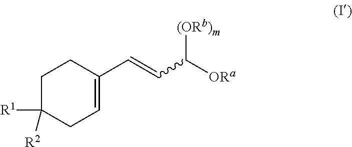

in the form of any one of its stereoisomers or a mixture thereof, and wherein m, R^{a}, R^{b}, R¹ and R² have the same meaning as defined in claim 1;
   and said compound of formula (II) is of formula

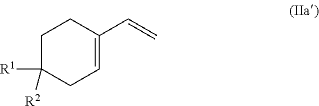

in the form of any one of its stereoisomers or a mixture thereof, and wherein each R¹ and R² have the same meaning as defined in claim 1.
According to any embodiment of the invention, the compound formula (I) is of formula

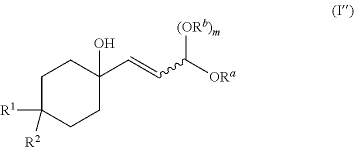

in the form of any one of its stereoisomers or a mixture thereof, and wherein m, R^{a}, R^{b}, R¹ and R² have the same meaning as defined in claim 1;
   and said compound of formula (II) is of formula

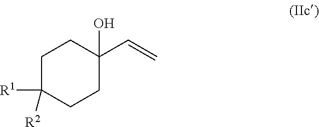

in the form of any one of its stereoisomers or a mixture thereof, and wherein each R¹ and R² have the same meaning as defined in claim 1.

According to any embodiment of the invention, R¹ may be a C₁₋₄ alkyl group or a C₂₋₄ alkenyl group. Particularly, R¹ may be a methyl, an ethyl, a propyl, an iso-propyl, an iso-butyl, a sec-butyl, a tert-butyl or a n-butyl group. Particularly, R¹ may be a methyl, an ethyl, a propyl, an iso-propyl, an iso-butyl, a sec-butyl or a n-butyl group. Even more particularly, R¹ may be a methyl group.

According to any embodiment of the invention, R² may be a hydrogen atom or a C₁₋₃ alkyl group or a C₂₋₃ alkenyl group. Particularly, R² may be a hydrogen atom, a methyl, an ethyl, a propyl or an iso-propyl group. Even more particularly, R² may be a methyl group.

According to any embodiment of the invention, when m is 1, R^{a} and R^{b}, independently from each other, may be a C₁₋₄ alkyl group, a C(═O)—R^{c} group wherein R^{c} may be a C₁₋₄ alkyl group; or R^{a} and R^{b} may be taken together and may be a C₂₋₆ alkanediyl group. Particularly, when m is 1, R^{a} and R^{b}, independently from each other, may be a C₁₋₃ alkyl group, a C(═O)—R^{c} group wherein R^{c} may be a C₁₋₃ alkyl group; or R^{a} and R^{b} may be taken together and may be a C₂₋₄ alkanediyl group. Particularly, when m is 1, R^{a} and R^{b}, independently from each other, may be a C₁₋₂ alkyl group, a C(═O)—R^{c} group wherein R^{c} may be a C₁₋₂ alkyl group; or R^{a} and R^{b} may be taken together and may be a (CH₂)˜ group wherein n may be 2 or 3; preferably n may be 2. Particularly, when m is 1, R^{a} and R^{b}, independently from each other, may be an ethyl group, a C(═O)—R^{c} group wherein R^{c} is a methyl group; or R^{a} and R^{b} are taken together and represent a (CH₂)₂ group.

According to any embodiment of the invention, when m is 0, R^{a} is a C₁₋₄ alkyl group, a trimethyl silyl group, a C(═O)—R^{c} group, a C(═O)—OR^{c} group, a CH₂(OR^{c}) group, a CH(OR^{c})CH₃ group wherein R^{c} is a C₁₋₄ alkyl group. Particularly, when m is 0, R^{a} may be a C₁₋₄ alkyl group, a C(═O)—R^{c} group wherein R^{c} may be a C₁₋₄ alkyl group. Particularly, when m is 0, R^{a} may be a C₁₋₃ alkyl group, a C(═O)—R^{c} group wherein R^{c} may be a C₁₋₃ alkyl group. Particularly, when m is 0, R^{a} may be a C₁₋₂ alkyl group, a C(═O)—R^{c} group wherein R^{c} may be a C₁₋₂ alkyl group. Particularly, when m is 0, R^{a} may be a C(═O)—R^{c} group wherein R^{c} is a methyl group.

According to any embodiment of the invention, m may be 1.

According to any embodiment of the invention, R^{d} may be a hydrogen atom.

Non-limiting examples of suitable compounds of formula (I) may include 1-(3,3-diethoxyprop-1-en-1-yl)-4,4-dimethylcyclohexan-1-ol, 3-(1-hydroxy-4,4-dimethylcyclohexyl)allyl acetate, 1-(3-butoxyprop-1-en-1-yl)-4,4-dimethylcyclohexan-1-ol, 1-(3-(1-butoxyethoxy)prop-1-en-1-yl)-4,4-dimethylcyclohexan-1-ol, 1-(3-hydroxyprop-1-en-1-yl)-4,4-dimethylcyclohexan-1-ol, 3-(1-hydroxy-4,4-dimethylcyclohexyl)acrylaldehyde, 3-(1-hydroxy-4,4-dimethylcyclohexyl)prop-2-ene-1,1-diyl diacetate, 1-(2-(1,3-dioxolan-2-yl)vinyl)-4,4-dimethylcyclohexan-1-ol, 3-(4,4-dimethylcyclohex-1-en-1-yl)prop-2-ene-1,1-diyl diacetate, 1-(3,3-diethoxyprop-1-en-1-yl)-4,4-dimethylcyclohex-1-ene, 2-(2-(4,4-dimethylcyclohex-1-en-1-yl)vinyl)-1,3-dioxolane, 4-butyl-1-(3,3-diethoxyprop-1-en-1-yl)cyclohexan-1-ol, 3-(4-butyl-1-hydroxycyclohexyl)prop-2-ene-1,1-diyl diacetate, 1-(2-(1,3-dioxolan-2-yl)vinyl)-4-butylcyclohexan-1-ol, 4-butyl-1-(3,3-diethoxyprop-1-en-1-yl)cyclohex-1-ene, 3-(4-butylcyclohex-1-en-1-yl)prop-2-ene-1,1-diyl diacetate, 2-(2-(4-butylcyclohex-1-en-1-yl)vinyl)-1,3-dioxolane, 3-(4-butyl-1-hydroxycyclohexyl)acrylaldehyde, 4-butyl-1-(3-hydroxyprop-1-en-1-yl)cyclohexan-1-ol, 3-(4-butyl-1-hydroxycyclohexyl)allyl acetate, 1-(3-butoxyprop-1-en-1-yl)-4-butylcyclohexan-1-ol, 1-(3-(1-butoxyethoxy)prop-1-en-1-yl)-4-butylcyclohexan-1-ol, 1-(3,3-diethoxyprop-1-en-1-yl)-3-isopropylcyclohexan-1-ol, 3-(1-hydroxy-3-isopropylcyclohexyl)prop-2-ene-1,1-diyl diacetate, 1-(2-(1,3-dioxolan-2-yl)vinyl)-3-isopropylcyclohexan-1-ol, 3-(5-isopropylcyclohex-1-en-1-yl)prop-2-ene-1,1-diyl diacetate, 3-(3-isopropylcyclohex-1-en-1-yl)prop-2-ene-1,1-diyl diacetate, 1-(3,3-diethoxyprop-1-en-1-yl)-5-isopropylcyclohex-1-ene, 1-(3,3-diethoxyprop-1-en-1-yl)-3-isopropylcyclohex-1-ene, 2-(2-(5-isopropylcyclohex-1-en-1-yl)vinyl)-1,3-dioxolane, 2-(2-(3-isopropylcyclohex-1-en-1-yl)vinyl)-1,3-dioxolane, 3-(1-hydroxy-3-isopropylcyclohexyl)acrylaldehyde, 1-(3-hydroxyprop-1-en-1-yl)-3-isopropylcyclohexan-1-ol, 3-(1-hydroxy-3-isopropylcyclohexyl)allyl acetate, 1-(3-butoxyprop-1-en-1-yl)-3-isopropylcyclohexan-1-ol, 1-(3-(1-butoxyethoxy)prop-1-en-1-yl)-3-isopropylcyclohexan-1-ol, 1-(5,5-diethoxy-2-methylpenta-1,3-dien-1-yl)-4-methylbenzene, 5,5-diethoxy-2-methyl-1-(p-tolyl)pent-3-en-2-ol, 5,5-diethoxy-2-methyl-1-(p-tolyl)pent-3-en-1-yl acetate, 5,5-diethoxy-2-methyl-1-(p-tolyl)pent-3-en-1-ol, 4-methyl-5-(p-tolyl)penta-2,4-diene-1,1-diyl diacetate, 4-hydroxy-4-methyl-5-(p-tolyl)pent-2-ene-1,1-diyl diacetate, 5-hydroxy-4-methyl-5-(p-tolyl)pent-2-ene-1,1-diyl diacetate, 4-methyl-5-(p-tolyl)pent-2-ene-1,1,5-triyl triacetate, 1-(tert-butyl)-4-(3,3-diethoxyprop-1-en-1-yl)benzene, 1-(4-(3,3-diethoxyprop-1-en-1-yl)phenyl)-2-methylpropan-2-ol, 3-(4-(2-hydroxy-2-methylpropyl)phenyl)prop-2-ene-1,1-diyl diacetate, or 3-(4-(tert-butyl)phenyl)prop-2-ene-1,1-diyl diacetate.

Non-limiting examples of suitable compounds of formula (II) may include 4,4-dimethyl-1-vinylcyclohexan-1-ol, 4-butyl-1-vinylcyclohexan-1-ol, 3-isopropyl-1-vinylcyclohexan-1-ol, 4,4-dimethyl-1-vinylcyclohex-1-ene, 4-butyl-1-vinylcyclohex-1-ene, 5-isopropyl-1-vinylcyclohex-1-ene, 3-isopropyl-1-vinylcyclohex-1-ene, 1-methyl-4-(2-methylbuta-1,3-dien-1-yl)benzene, 2-methyl-1-(p-tolyl)but-3-en-2-ol, 2-methyl-1-(p-tolyl)but-3-en-1-yl acetate, 2-methyl-1-(4-vinylphenyl)propan-2-ol or 1-(tert-butyl)-4-vinylbenzene.

Non-limiting examples of suitable compounds of formula (III) may include 3,3-diethoxyprop-1-ene, 1,1,4,4-tetraethoxybut-2-ene, 3,3-Dimethoxy-1-propene, 2-vinyl-1,3-dioxolane, 1,2-di(1,3-dioxolan-2-yl)ethene, allyl acetate, allyl methyl carbonate, 1-(allyloxy)butane, 1-(1-(allyloxy)ethoxy)butane, 1,4-dibutoxybut-2-ene, 6,13-dimethyl-5,7,12,14-tetraoxaoctadec-9-ene, but-2-ene-1,4-diyl diacetate or prop-2-ene-1,1-diyl diacetate.

The compounds of formula (II) and (III) are commercially available compounds or can be prepared by several methods, e.g. 1-methyl-4-(2-methylbuta-1,3-dien-1-yl)benzene may be prepared following the protocol reported in J. Am. Chem. Soc. 2020, 142, 9932-9937 or 4,4-dimethyl-1-vinyl-cyclohexanol may be prepared according to Angew. Chem. Int. Ed. 2009, 48, 3146 from the commercial ketone (Vinyl Grignard addition) and may be further converted into 4,4-dimethyl-1-vinylcyclohex-1-ene by an acid catalysed dehydration according to Angew. Chem. Int. Ed., 2009, 48, 3146.

According to any embodiments of the invention, the metathesis catalyst may be a cross-metathesis catalyst. In particular, the metathesis catalyst may be a Ruthenium-based catalyst, Molybdenum-based catalyst, Rhenium-based catalyst or Tungsten-based catalyst. Particularly, the metathesis catalyst may be a Ruthenium-based catalyst. The Ruthenium-based metathesis catalyst may be a Ruthenium(II) carbenoid complex. The nature and type of Ruthenium-based metathesis catalyst used in the invention's process do not warrant a more detailed description here, which in any case would not be exhaustive, the skilled person being able to select them on the basis of his general knowledge. Said catalysts are in any case listed in reference texts such as Grubbs, R. H. Handbook of Metathesis; Wiley-VCH: New York, 2003; 1204 pages, 3 volumes, The Strem Chemiker—Vol. XXVIII No. 1, June, 2015, pages 1-24. Booklet Strem Metathesis Catalysts February 2020, R. H. Grubbs, A. G. Wenzel, D. J. OLeary, E. Khosravi, Handbook of Metathesis, Wiley-VCH, Weinheim, 2015, K. Grela, Olefin Metathesis: Theory and Practice, Wiley, Hoboken, 2014 or in other works of a similar nature, as well as in the abundant patent literature in the field of metathesis processes. Non-limiting examples of suitable metathesis catalyst may include (1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), (1,3-bis(2,6-diisopropylphenyl)imidazolidin-2-ylidene)diiodo(2-((1-(methoxy(methyl)amino)-1-oxopropan-2-yl)oxy)benzylidene)ruthenium(II), (1,3-dimesitylimidazolidin-2-ylidene)dichloro(2-isopropoxy-5-nitrobenzylidene)ruthenium(II), Dichloro[1,3-bis(2,4,6-trimethylphenyl)-2-imidazolidinylidene](2-isopropoxyphenylmethylene)ruthenium(II), Benzylidene-bis(tricyclohexylphosphine)dichlororuthenium, Dichloro[1,3-bis(2,6-isopropylphenyl)-2-imidazolidinylidene](2-isopropoxyphenylmethylene)ruthenium(II), [1,3-Bis(2,6-di-i-propylphenyl)imidazolidin-2-ylidene)(2-i-propoxy-5-nitrobenzylidene)ruthenium(II) diiodide, 1,3-Bis(2,6-di-i-propylphenyl)imidazolidin-2-ylidene)(2-i-propoxy-5-nitrobenzylidene) ruthenium(II) dichloride, (1,3-Dimesitylimidazolidin-2-ylidene)diiodo(2-isopropoxy-5-nitrobenzylidene)ruthenium(II), Bis(1-(2,6-diethylphenyl)-3,5,5-trimethyl-3-phenylpyrrolidin-2-ylidene)(3-phenyl-1H-inden-1-ylidene)ruthenium(II) dichloride, (1-(2,6-diethylphenyl)-3,5,5-trimethyl-3-phenylpyrrolidin-2-ylidene)dichloro(2-isopropoxy-5-nitrobenzylidene)ruthenium(II), (1-(2,6-diethylphenyl)-3,5,5-trimethyl-3-phenylpyrrolidin-2-ylidene)diiodo(2-isopropoxy-5-nitrobenzylidene)ruthenium(II), Dichloro[1,3-bis(2,4,6-trimethylphenyl)-2-imidazolidinylidene](benzylidene)(tricyclohexylphosphine)ruthenium(II), Dichloro[1,3-bis(2-methylphenyl)-2-imidazolidinylidene](2-isopropoxyphenylmethylene)ruthenium(II), (2-(2,6-Diethylphenyl)-3,3-dimethyl-2-azaspiro[4.5]decan-1-yl)(2-isopropoxy-5-nitrobenzylidene)ruthenium(II) dichloride, [2-(1-Methylethoxy-O)phenylmethyl-C](nitrato-O,O′){rel-(2R,5R,7R)-adamantane-2,1-diyl[3-(2,4,6-trimethylphenyl)-1-imidazolidinyl-2-ylidene]}ruthenium, Dichloro(2-isopropoxyphenylmethylene) (tricyclohexylphosphine)ruthenium(II), Dichloro(3-phenyl-1H-inden-1-ylidene)bis(tricyclohexylphosphine)ruthenium(II), 1,3-Bis(2,4,6-trimethylphenyl)-4,5-dihydroimidazol-2-ylidene[2-(i-propoxy)-5-(N,N-dimethylaminosulfonyl)phenyl]methyleneruthenium(II) dichloride (resin supported), Tricyclohexylphosphine[1,3-bis(2,4,6-trimethylphenyl)imidazol-2-ylidene][3-phenyl-1H-inden-1-ylidene]ruthenium(II) dichloride, Dichloro[1,3-bis(2,4,6-trimethylphenyl)-2-imidazolidinylidene](3-phenyl-1H-inden-1-ylidene)(tricyclohexylphosphine)ruthenium(II), [1,3-Bis(2,4,6-trimethylphenyl)-2-imidazolidinylidene]-[2-[[(4-methylphenyl)imino]methyl]-4-nitrophenolyl]-[3-phenyl-1H-inden-1-ylidene]ruthenium(II) chloride, Dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II), Dichloro[1,3-Bis(2-methylphenyl)-2-imidazolidinylidene](benzylidene)(tricyclohexylphosphine)ruthenium(II), Dichloro[1,3-bis(2,4,6-trimethylphenyl)-2-imidazolidinylidene](3-methyl-2-butenylidene) (tricyclohexylphosphine)ruthenium(II), Dichloro[1,3-bis(2,4,6-trimethylphenyl)-2-imidazolidinylidene](3-methyl-2-butenylidene)(dipyridine)ruthenium(II), [1,3-Bis(2,4,6-trimethylphenyl)-4-[(trimethylammonio)methyl]imidazolidin-2-ylidene]-(2-i-propoxy-5-nitrobenzylidene)dichlororuthenium(II) chloride, Dichloro[1-(2,6-diisopropylphenyl)-2,2,4-trimethyl-4-phenyl-5-pyrrolidinylidene](2-isopropoxyphenylmethylene)ruthenium(II), [1,3-Bis(2,6-diisopropylphenyl)-2-imidazolidinylidene]dichloro[(2-isopropoxy)(5-trifluoroacetamido)benzylidene]ruthenium(II), Dichloro[1,3-bis(2,4,6-trimethylphenyl)-2-imidazolidinylidene](2-methoxyphenylmethylene)ruthenium(II), Dichloro[1,3-bis(2,4,6-trimethylphenyl)-2-imidazolidinylidene](3-methyl-2-butenylidene)(dipyridine)ruthenium(II), Dichloro[1,3-bis(2,4,6-trimethylphenyl)-2-imidazolidinylidene](3-phenyl-1H-inden-1-ylidene)(diphenylmethoxyphosphine)ruthenium(II), Dichloro[1,3-bis(2,4,6-trimethylphenyl)-2-imidazolidinylidene](3-phenyl-1H-inden-1-ylidene)(triphenylphosphine)ruthenium(II), Dichloro[1,3-bis(2,6-diisopropylphenyl)-2-imidazolidinylidene](benzylidene)(tricyclohexylphosphine)ruthenium(II), Dichloro[1,3-bis(2,4,6-trimethylphenyl)-2-imidazolidinylidene](3-phenyl-1H-inden-1-ylidene)diphenylphenoxyphosphine]ruthenium(II), Dichloro[1,3-bis(2-isopropylphenyl)-2-imidazolidinylidene](2-isopropoxyphenylmethylene)ruthenium(II), Dichloro[1-(2,4,6-trimethylphenyl)-2,2,4-trimethyl-4-phenyl-5-pyrrolidinylidene](2-isopropoxyphenylmethylene)ruthenium(II), Dichloro[1-(2,6-diisopropylphenyl)-2,2,4-trimethyl-4-phenyl-5-pyrrolidinylidene](2-isopropoxyphenylmethylene)ruthenium(II), Dichloro(3-methyl-2-butenylidene)bis(tricyclohexylphosphine)ruthenium(II), Dichloro[1,3-bis(2,4,6-trimethylphenyl)-2-imidazolidinylidene][2-(N,N-dimethylamino)-phenylmethylene]ruthenium(II), Dichloro[1,3-bis(2,6-diisopropylphenyl)-2-imidazolidinylidene][2-(N,N-dimethylamino)-phenylmethylene]ruthenium(II), Dichloro[1,3-bis(2,4,6-trimethylphenyl)-2-imidazolidinylidene](benzylidene)(tri-nbutylphosphine) ruthenium(II), Dichlorobis[1,3-bis(2,4,6-trimethylphenyl)-2-imidazolidinylidene](benzylidene)ruthenium(II), Dichlorobis[1,3-bis(2,4,6-trimethylphenyl)-2-imidazolidinylidene](2-isopropoxyphenylmethylene)ruthenium(II), (1,3-di-o-tolylimidazolidin-2-ylidene)dichloro(2-isopropoxy-5-nitrobenzylidene)ruthenium(II), Dichloro(2-isopropoxy-5-nitrophenylmethylene)(tricyclohexylphosphine)ruthenium(II), 1,3-Bis(2,4,6-trimethylphenylimidazolidin-2-ylidene)chloro(tricyclohexylphosphine)-(2-oxobenzylidene)ruthenium(II), 1,3-Bis(2,4,6-trimethylphenylimidazolidin-2-ylidene)chloro(tricyclohexylphosphine)-(2-oxo-5-nitrobenzylidene)ruthenium(II), 1,3-Bis(2,4,6-trimethylphenylimidazolidin-2-ylidene)iodo(tricyclohexylphosphine)-(2-oxobenzylidene)ruthenium(II), (1,3-dimesitylimidazolidin-2-ylidene)dichloro(2-((2-ethoxy-2-oxoethylidene)amino)benzylidene)ruthenium(II), (1,3-bis(2,6-diisopropylphenyl)imidazolidin-2-ylidene)dichloro(2-((2-ethoxy-2-oxoethylidene)amino)benzylidene)ruthenium(II), (4-((4-ethyl-4-methylpiperazin-1-ium-1-yl)methyl)-1,3-dimesitylimidazolidin-2-ylidene)dichloro(2-isopropoxybenzylidene)ruthenium(II) chloride, (4-((4-ethyl-4-methylpiperazin-1-ium-1-yl)methyl)-1,3-dimesitylimidazolidin-2-ylidene)dichloro(2-isopropoxybenzylidene)ruthenium(II) hexafluorophosphate, (1,3-dimesityl-4-((trimethylammonio)methyl)imidazolidin-2-ylidene)dichloro(2-isopropoxybenzylidene)ruthenium(II) hexafluorophosphate, (1,3-dimesityl-4-((trimethylammonio)methyl)imidazolidin-2-ylidene)dichloro(2-isopropoxy-5-nitrobenzylidene)ruthenium(II), (1,3-dimesityl-4-((trimethylammonio)methyl)imidazolidin-2-ylidene)dichloro(2-isopropoxybenzylidene)ruthenium(II) tetrafluoroborate, (1,3-bis(2,6-diisopropylphenyl)-4-((4-ethyl-4-methylpiperazin-1-ium-1-yl)methyl)imidazolidin-2-ylidene)dichloro(2-isopropoxybenzylidene)ruthenium(II) chloride, (1,3-bis(2,6-diisopropylphenyl)-4-((4-ethyl-4-methylpiperazin-1-ium-1-yl)methyl)imidazolidin-2-ylidene)dichloro(2-isopropoxybenzylidene)ruthenium(II) hexafluorophosphate, bis(2-(2,6-diethylphenyl)-3,3-dimethyl-2-azaspiro[4.5]decan-1-ylidene)dichloro(3-phenyl-1H-inden-1-ylidene)ruthenium(II) dichloromethane complex, [1,3-Bis(2,6-di-i-propylphenyl)imidazolidin-2-ylidene) (tricyclohexylphosphine)-(2-oxobenzylidene)ruthenium(II) chloride, Bis(tricyclohexylphosphine)[(phenylthio)methylene]ruthenium(II) dichloride, [1,3-Bis(2,4,6-trimethylphenyl)-2-imidazolidinylidene]-[2-[[(2-methylphenyl)imino]methyl]phenolyl]-[3-phenyl-1H-inden-1-ylidene]ruthenium(II) chloride, 1,3-Bis(2,4,6-trimethylphenyl)-2-imidazolidinylidene)(3-phenyl-1H-inden-1-ylidene)(4,5-dichloro-1,3-diethyl-1,3-dihydro-2H-imidazol-2-ylidene)ruthenium(II) dichloride, 3-Phenyl-1H-inden-1-ylidene[bis(i-butylphobane)]ruthenium(II) dichloride, {[2-(i-Propoxy)-5-(N,N-dimethylaminosulfonyl)phenyl]methylene}(tricyclohexylphosphine) ruthenium(II) dichloride, Tricyclohexylphosphine[1,3-bis(2,4,6-trimethylphenyl)-4,5-dihydroimidazol-2-ylidene][(phenylthio)methylene]ruthenium(II) dichloride, Tricyclohexylphosphine[1,3-bis(2,4,6-trimethylphenyl)imidazol-2-ylidene][2-thienylmethylene]ruthenium(II) dichloride, Tricyclohexylphosphine[2,4-dihydro-2,4,5-triphenyl-3H-1,2,4-triazol-3-ylidene][2-thienylmethylene]ruthenium(II) dichloride, Tricyclohexylphosphine[4,5-dimethyl-1,3-bis(2,4,6-trimethylphenyl)imidazol-2-ylidene][2-thienylmethylene]ruthenium(II) dichloride, Tri(i-propoxy)phosphine(3-phenyl-1H-inden-1-ylidene)[1,3-bis(2,4,6-trimethylphenyl)-4,5-dihydroimidazol-2-ylidene]ruthenium (II) dichloride, Dichloro[1,3-bis(2,6-diisopropylphenyl)imidazolidin-2-ylidene][(5-(2-ethoxy-2-oxoethanamido))-(2-isopropoxy)benzylidene]ruthenium(II), Dichloro[1,3-bis(2,6-diisopropylphenyl)-2-imidazolidinylidene][(2-isopropoxy)(5-pentafluorobenzoylamino)benzylidene]ruthenium(II), Dichloro[1,3-bis(2,4,6-trimethylphenyl)-2-imidazolidinylidene]{[5-(2-ethoxy-2-oxoethanamido)]-2-isopropoxybenzylidene}ruthenium(II), Dichloro[1,3-bis(2,4,6-trimethylphenyl)-2-imidazolidinylidene][(2-isopropoxy)(5-pentafluorobenzoylamino)benzylidene]ruthenium(II), (1,3-Bis(2,6-diisopropylphenyl)imidazolidin-2-ylidene)dichloro(2-((1-(methoxy(methyl)amino)-3-methyl-I-oxobutan-2-yl)oxy)benzylidene)ruthenium(II), Dichloro[1,3-bis(2,4,6-trimethylphenyl)-2-imidazolidinylidene](3-phenyl-1H-inden-1-ylidene)(pyridyl)ruthenium(II), Dichloro[1,3-bis(2,4,6-trimethylphenyl)-2-imidazolidinylidene]{[2-(1-methylacetoxy)phenyl]methylene}ruthenium(II), Dichloro[1,3-bis(2,4,6-trimethylphenyl)-2-imidazolidinylidene][(2-isopropoxy)(5-trifluoroacetamido)benzylidene]ruthenium(II), Dichloro[1,3-bis(2,6-diisopropylphenyl)imidazolidin-2-ylidene][(5-isobutoxycarbonylamino)-(2-isopropoxy)benzylidene]ruthenium(II) or Dichloro[1,3-bis(2,6-diisopropylphenyl)-2-imidazolidinylidene](3-phenyl-1H-inden-1-ylidene)(triphenylphosphine)ruthenium(II). Even more particularly, the metathesis catalyst may be selected from the group consisting of (1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), (1,3-bis(2,6-diisopropylphenyl)imidazolidin-2-ylidene)diiodo(2-((1-(methoxy(methyl)amino)-1-oxopropan-2-yl)oxy)benzylidene)ruthenium(II), (1,3-dimesitylimidazolidin-2-ylidene)dichloro(2-isopropoxy-5-nitrobenzylidene)ruthenium(II), Dichloro[1,3-bis(2,4,6-trimethylphenyl)-2-imidazolidinylidene](2-isopropoxyphenylmethylene)ruthenium(II), Benzylidene-bis(tricyclohexylphosphine)dichlororuthenium, Dichloro[1,3-bis(2,6-isopropylphenyl)-2-imidazolidinylidene](2-isopropoxyphenylmethylene)ruthenium(II), [1,3-Bis(2,6-di-i-propylphenyl)imidazolidin-2-ylidene)(2-i-propoxy-5-nitrobenzylidene) ruthenium(II) diiodide, 1,3-Bis(2,6-di-i-propylphenyl)imidazolidin-2-ylidene)(2-i-propoxy-5-nitrobenzylidene) ruthenium(II) dichloride, (1,3-Dimesitylimidazolidin-2-ylidene)diiodo(2-isopropoxy-5-nitrobenzylidene)ruthenium(II), Bis(1-(2,6-diethylphenyl)-3,5,5-trimethyl-3-phenylpyrrolidin-2-ylidene)(3-phenyl-1H-inden-1-ylidene)ruthenium(II) dichloride, (1-(2,6-diethylphenyl)-3,5,5-trimethyl-3-phenylpyrrolidin-2-ylidene)dichloro(2-isopropoxy-5-nitrobenzylidene)ruthenium(II), (1-(2,6-diethylphenyl)-3,5,5-trimethyl-3-phenylpyrrolidin-2-ylidene)diiodo(2-isopropoxy-5-nitrobenzylidene)ruthenium(II), Dichloro[1,3-bis(2-methylphenyl)-2-imidazolidinylidene](2-isopropoxyphenylmethylene)ruthenium(II), 1,3-Bis(2,4,6-trimethylphenyl)-4,5-dihydroimidazol-2-ylidene[2-(i-propoxy)-5-(N,N-dimethylaminosulfonyl)phenyl]methyleneruthenium(II) dichloride (resin supported), Dichloro[1,3-bis(2,4,6-trimethylphenyl)-2-imidazolidinylidene](3-phenyl-1H-inden-1-ylidene)(tricyclohexylphosphine)ruthenium(II), [1,3-Bis(2,4,6-trimethylphenyl)-2-imidazolidinylidene]-[2-[[(4-methylphenyl)imino]methyl]-4-nitrophenolyl]-[3-phenyl-1H-inden-1-ylidene]ruthenium(II) chloride, Dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II), Dichloro[1-(2,6-diisopropylphenyl)-2,2,4-trimethyl-4-phenyl-5-pyrrolidinylidene](2-isopropoxyphenylmethylene)ruthenium(II), [1,3-Bis(2,6-diisopropylphenyl)-2-imidazolidinylidene]dichloro[(2-isopropoxy)(5-trifluoroacetamido)benzylidene]ruthenium(II), Dichloro[1,3-bis(2,6-diisopropylphenyl)-2-imidazolidinylidene](benzylidene)(tricyclohexylphosphine)ruthenium(II), Dichloro[1-(2,4,6-trimethylphenyl)-2,2,4-trimethyl-4-phenyl-5-pyrrolidinylidene](2-isopropoxyphenylmethylene)ruthenium(II), Dichloro[1-(2,6-diisopropylphenyl)-2,2,4-trimethyl-4-phenyl-5-pyrrolidinylidene](2-isopropoxyphenylmethylene)ruthenium(II), Dichloro[1,3-bis(2,6-diisopropylphenyl)-2-imidazolidinylidene][(2-isopropoxy)(5-pentafluorobenzoylamino)benzylidene]ruthenium(II), Dichloro[1,3-bis(2,4,6-trimethylphenyl)-2-imidazolidinylidene]{[5-(2-ethoxy-2-oxoethanamido)]-2-isopropoxybenzylidene}ruthenium(II), Dichloro[1,3-bis(2,4,6-trimethylphenyl)-2-imidazolidinylidene][(2-isopropoxy)(5-pentafluorobenzoylamino)benzylidene]ruthenium(II), (1,3-Bis(2,6-diisopropylphenyl)imidazolidin-2-ylidene)dichloro(2-((1-(methoxy(methyl)amino)-3-methyl-1-oxobutan-2-yl)oxy)benzylidene)ruthenium(II), Dichloro[1,3-bis(2,4,6-trimethylphenyl)-2-imidazolidinylidene](3-phenyl-1H-inden-1-ylidene)(pyridyl)ruthenium(II), Dichloro[1,3-bis(2,4,6-trimethylphenyl)-2-imidazolidinylidene]1[(2-isopropoxy)(5-trifluoroacetamido)benzylidene]ruthenium(II), Dichloro[1,3-bis(2,6-diisopropylphenyl)imidazolidin-2-ylidene][(5-isobutoxycarbonylamino)-(2-isopropoxy)benzylidene]ruthenium(II) and Dichloro[1,3-bis(2,6-diisopropylphenyl)-2-imidazolidinylidene](3-phenyl-1H-inden-1-ylidene)(triphenylphosphine)ruthenium(II).

The metathesis catalyst can be added into the reaction medium of the invention's process in a large range of concentrations. As non-limiting examples, one can cite as catalyst concentration values those ranging from 2 ppm to 200000 ppm, relative to the total amount of compound of formula (II). Preferably, the catalyst concentration will be comprised between 10 ppm to 50000 ppm, or even between 30 ppm and 2000 ppm. It goes without saying that the process works also with more catalyst. However the optimum concentration of the catalyst will depend, as the person skilled in the art knows, on the nature of the latter, on the nature of the substrates, on the temperature and on the desired time of reaction.

According to any embodiments of the invention, a scavenger may be added to the invention process. In particular, the scavenger may be added after 30 minutes, 1 hour, 2 hours, 3 hours, 10 hours, 20 hours, 24 hours, 36 hours. Non-limiting example of suitable scavengers include amines, 1,4-Bis(2-isocyanopropyl)piperazine, pyridines, imidazoles nitriles (polynitriles), sulfoxides such as DMSO, amides, thiols, Pb(OAc)₄, 2-mercaptonicotinic acid (MNA), cysteine, chelating phosphines, triphenylphosphine oxide (TPPO), di(ethylene glycol) vinylether, phosphanetriyltrimethanol (THMP), Na₂S₂O₅, H₂O₂ or silica-bases heterogenous particles.

The scavenger can be added into the reaction medium of the invention's process in a large range of concentrations. As non-limiting examples, one can cite as scavenger concentration values those ranging from 5 equivalents to 10 equivalents relative to the amount of the metathesis catalyst. It goes without saying that the optimum concentration of scavenger will depend, as the person skilled in the art knows, on the nature of the latter, on the nature of the substrate, of the temperature and on the catalyst used during the process, as well as the desired time of reaction.

The compound of formula (III) can be added into the reaction medium of the invention's process in a large range of concentrations. As non-limiting examples, one can cite as compound of formula (III) concentration values those ranging from 0.5 equivalents to 50 equivalents, or even between 1 equivalent to 5 equivalents, relative to the amount of compound of formula (II). It goes without saying that the optimum concentration of compound of formula (III) will depend, as the person skilled in the art knows, on the nature of the latter, on the nature of compound of formula (II), of the temperature and on the catalyst used during the process, as well as the desired time of reaction.

The invention's process is carried out under batch, semi-batch or continuous conditions.

The reaction can be carried out in the absence of a solvent. When a solvent is required or used for practical reasons, then any solvent current in metathesis reactions can be used for the purposes of the invention. Non-limiting examples include C₆₋₁₀ aromatic solvents such as toluene or xylene; C₅₋₁₂ hydrocarbon solvents such as hexane, heptane, or cyclohexane; C₄₋₈ ethers such as tetrahydrofuran, 2-MeTHF or MTBE; C₄₋₁₀ esters such as ethyl acetate and i-PrOAc; C₁₋₂ chlorinated hydrocarbon, such as dichloromethane, dichloroethane, or chlorobenzene; C₂₋₆ primary or secondary alcohols, such as isopropanol, methanol or ethanol; C₂-6 polar solvents such as acetone or HOAc and water (neutral/acidic); or mixtures thereof. In particular said solvent can be a solvent such as dichloromethane, toluene or no solvent. The choice of the solvent is a function of the nature of the metathesis catalyst and the compound of formula (II) and (III). The person skilled in the art is well able to select the solvent most convenient in each case to optimize the invention's process.

The temperature of the invention's process may be comprised between 20° C. and 110° C., preferably, in the range comprised between 20° C. and 80° C. more preferably in the range comprised between 20° C. and 50° C. Of course, a person skilled in the art is also able to select the preferred temperature as a function of the melting and boiling point of the starting and final products as well as the desired time of reaction or conversion.

The invention's process may be performed under atmospheric pressure or reduced pressure. The invention's process may be performed under inert atmosphere such as nitrogen and/or argon.

The invention's process may lead to the formation of side products such as dimer of compound of formula (II) such as 1,1′-(ethene-1,2-diyl)bis(4,4-dimethylcyclohexan-1-ol), 1,2-bis(4,4-dimethylcyclohex-1-en-1-yl)ethene, 4,4′-(2,5-dimethylhexa-1,3,5-triene-1,6-diyl)bis(methylbenzene), 2,5-dimethyl-1,6-di-p-tolylhex-3-ene-1,6-diyl diacetate, 2,5-dimethyl-1,6-di-p-tolylhex-3-ene-2,5-diol, 1,1′-(ethene-1,2-diylbis(4,1-phenylene))bis(2-methylpropan-2-ol) and 1,2-bis(4-(tert-butyl)phenyl)ethene or dimer of compound of formula (III) such as 1,1,4,4-tetraethoxybut-2-ene, 1,1,4,4-tetramethoxybut-2-ene, but-2-ene-1,1,4,4-tetrayl tetraacetate and 1,2-di(1,3-dioxolan-2-yl)ethene, but-2-ene-1,4-diyl diacetate, 1,4-dibutoxybut-2-ene, 6,13-dimethyl-5,7,12,14-tetraoxaoctadec-9-ene. Most of the side products formed may be recycled in the invention's process. In addition, unreacted starting materials may be also recycled in the invention's process.

The invention's process may lead to the formation of the aldehyde of formula

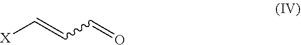

in the form of any one of its stereoisomers or a mixture thereof, and wherein X as the same meaning as defined above.

According to any embodiment of the invention, the compound of formula (I) may be further converted to compound of formula (V)

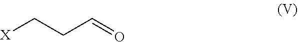

in the form of any one of its stereoisomers or a mixture thereof, and wherein X is a group of formula a) or b) having the same meaning as defined above. When the cross metathesis step is carried out between the compound of formula (IIa) or (IIb) with Y being a —CH═CR¹¹— wherein R¹¹ is a hydrogen atom or a methyl or ethyl group and compound of formula (III) wherein m is 1, said compound of formula (V) may be prepared via a process comprising a deprotection and hydrogenation step. For the sake of clarity, by the expression “comprising a deprotection and hydrogenation step”, it is meant that the deprotection reaction and the hydrogenation reaction may be performed in any order. In other words, the invention process may comprise a deprotection step followed by an hydrogenation step or the invention process may comprise an hydrogenation step followed by a deprotection step.

According to a particular embodiment, the process for the preparation of a compound of formula (V) as defined above may comprises the step of
i) A cross metathesis between compound of formula (IIa) or (IIb) with Y being a —CH═CR¹¹— wherein R¹¹ is a hydrogen atom or a methyl or ethyl group and compound of formula (III), wherein m is 1 and R^{a}, R^{b} and R^{d} have the same meaning as defined above, to obtain compound of formula (I) wherein X is a group of formula a) or b) having the same meaning as defined above;
ii) A deprotection step to obtain compound of formula (IV) wherein X is a group of formula a) or b) having the same meaning as defined above; and
iii) A hydrogenation step to obtain compound of formula (V).

According to another particular embodiment, the process for the preparation of a compound of formula (V) as defined above may comprises the step of
i) A cross metathesis between compound of formula (IIa) or (IIb) with Y being a —CH═CR¹¹— wherein R¹¹ is a hydrogen atom or a methyl or ethyl group and compound of formula (III), wherein m is 1 and R^{a}, R^{b} and R^{d} have the same meaning as defined above, to obtain compound of formula (I) wherein X is a group of formula a) or b) having the same meaning as defined above;
ii) A hydrogenation step to obtain compound of formula

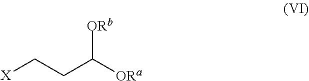

Wherein X is a group of formula a) or b) having the same meaning as defined above and R^{a} and R^{b} have the same meaning as define above; and
iii) A deprotection step to obtain compound of formula (V).

When the cross metathesis step is carried out between the compound of formula (IIc) or (IIb) with Y being a —CHR¹²—CHR¹¹— or —CH₂—C(OH)R¹¹— group wherein R¹¹ is a hydrogen atom or a methyl or ethyl group and R¹² is a hydroxy or a acetate group and compound of formula (III) wherein m is 1, the process for the preparation of a compound of formula (V) as defined above further comprises an elimination/dehydration step carried out before or after the deprotection step, particularly before the deprotection step. Particularly, the deprotection and elimination/dehydration steps may be carried out in one pot.

According to any embodiment of the invention, the deprotection, the hydrogenation and, optionally, the elimination/dehydration steps to form a compound of formula (V) may be carried out under normal conditions known by the person skilled in the art. A person skilled in the art is able to select the most suitable conditions to perform said transformations.

When the cross metathesis step is carried out between the compound of formula (II) and compound of formula (III) wherein m is 0, said compound of formula (V) as defined above may be prepared via a process comprising a deprotection and an isomerisation step. For the sake of clarity, by the expression “comprising a deprotection and an isomerisation step”, it is meant that the deprotection reaction and the isomerisation reaction may be performed in any order. In other words, the invention process may comprise a deprotection step followed by an isomerisation step or the invention process may comprise an isomerisation step followed by a deprotection step.

According to a particular embodiment, the process for the preparation of a compound of formula (V) as defined above may comprises the step of
i) A cross metathesis between compound of formula (II) and compound of formula (III), wherein m is 0 and R^{a} and R^{d} have the same meaning as defined above,
ii) a deprotection step to obtain compound of formula

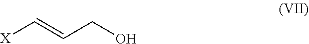

Wherein X is a group of formula a) or b) having the same meaning as defined above; and
iii) an isomerisation step to obtain compound of formula (V).

When the cross metathesis step is carried out between the compound of formula (II) being of formula (IIc) and compound of formula (III) wherein m is 0, the process for the preparation of a compound of formula (V) as defined above further comprises an elimination step carried out before or after the deprotection step, particularly before the deprotection step. Particularly, the deprotection and elimination steps may be carried out in one pot.

According to any embodiment of the invention, the isomerisation, the deprotection and, optionally, the elimination steps to form a compound of formula (V) may be carried out under normal conditions known by the person skilled in the art, i.e. such as for example, for isomerisation Journal of the American Chemical Society, 2006, 128(4), 1360-1370 or Chimia, 2009, 63(1-2), 35-37. A person skilled in the art is able to select the most suitable conditions to perform said transformations.

The compound of formula (I), (IV) and (VII) are, generally, novel compounds and present a number of advantages as explained above and shown in the Examples.

Therefore, another object of the present invention is a compound of formula

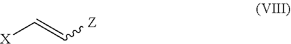

in the form of any one of its stereoisomers or a mixture thereof, and wherein X is a group of formula b) or d)

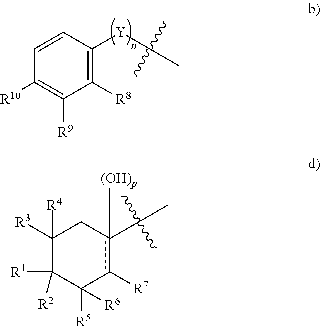

in the form of any one of its stereoisomers, and
wherein p is 0 when the dotted line is a carbon-carbon double bond and p is 1 when the dotted line is a carbon-carbon single bond;
each R¹, R², R³, R⁴, R⁵, R⁶ and R⁷, independently from each other, represent a hydrogen atom, a C₁₋₆ alkyl group or a C₂₋₆ alkenyl group, each optionally substituted by a hydroxy or C₁₋₃ alkoxy group; or two groups among R¹, R², R³, R⁴, R⁵, R⁶ and R⁷ are taken together and form C₃₋₈ cycloalkyl or C₅₋₈ cycloalkenyl group, each optionally substituted by a hydroxy, C₁₋₃ alkyl or C₁₋₃ alkoxy group, and the others groups have the same meaning as defined above;
each R⁸, R⁹ and R¹⁰, independently from each other, represent a hydrogen atom, a C₁₋₆ alkyl group or a C₂₋₆ alkenyl group, each optionally substituted by a hydroxy or C₁₋₃ alkoxy group; or two groups among R⁸, R⁹ and R¹⁰ are taken together and form C₃₋₈ cycloalkyl or C₅₋₈ cycloalkenyl group, each optionally substituted by a hydroxy, C₁_3 alkyl or C₁₋₃ alkoxy group, and the other group has the same meaning as defined above;
n is 0 or 1;
Y is a —CH═CR¹¹—, —CHR¹²—CHR¹¹— or —CH₂—C(OH)R¹¹— group wherein R¹¹ is a hydrogen atom or a methyl or ethyl group and R¹² is a hydroxy or a acetate group;
Z is, when X is of formula d), a CHO group, a CH₂OH group or a CH(OR^{a})(OR^{b})ₘ group wherein m is 0 or 1; R^{a} is a C₁₋₄ alkyl group, a trimethyl silyl group, a C(═O)—R^{c} group, a C(═O)—OR^{c} group, a CH₂(OR^{c}) group, a CH(OR^{c})CH₃ group wherein R^{c} is a C₁₋₄ alkyl group; R^{b} is a C₁₋₄ alkyl group, a C(═O)—R^{c} group wherein R^{c} is a C₁₋₄ alkyl group; or R^{a} and R^{b} are taken together and represent a C₂₋₆ alkanediyl group; or
Z is, when X is of formula b), a CH(OR^{a})(OR^{b}) group wherein R^{a} and R^{b}, independently from each other, are a methyl group or a C₃₋₄ alkyl group, a C(═O)—R^{c} group wherein R^{c} is a C₁₋₄ alkyl group; or R^{a} and R^{b} are taken together and represent a C₃₋₆ alkanediyl group; and
provided that when X is a group of formula (d), then at least one group among R¹, R², R³, R⁴, R⁵, R⁶ and R⁷ is a C₁₋₆ alkyl group or a C₂₋₆ alkenyl group, each optionally substituted by a hydroxy or C₁₋₃ alkoxy group; provided that when X is a group of formula (d) and six groups among R¹, R², R³, R⁴, R⁵, R⁶ and R⁷ are a hydrogen atom, then the group among R¹, R², R³, R⁴, R⁵, R⁶ and R⁷ being not a hydrogen atom is not a methyl group; provided that when X is a group of formula (d) and R², R³, R⁴, R⁵, R⁶ and R⁷ are a hydrogen atom then R¹ is not a prop-1-en-2-yl group; and provided that 3-(4-(tert-butyl)cyclohex-1-en-1-yl)acrylaldehyde, 3-(4-(tert-butyl)-1-hydroxycyclohexyl)acrylaldehyde, 1-(tert-butyl)-4-(3,3-diethoxyprop-1-en-1-yl)benzene, 2-(4-(tert-butyl)styryl)-1,3-dioxolane, 3-(4-(tert-butyl)-1-hydroxycyclohexyl)allyl acetate, 3-(2-allyl-1-hydroxycyclohexyl)allyl acetate, 4-(tert-butyl)-1-(3-hydroxyprop-1-en-1-yl)cyclohexan-1-ol, 2-allyl-1-(3-hydroxyprop-1-en-1-yl)cyclohexan-1-ol, 2-(4-isopropylstyryl)-1,3-dioxane, 2-(4-isopropylstyryl)-4-methyl-1,3-dioxolane, 3-(2-(buta-1,3-dien-1-yl)-1-hydroxycyclohexyl)acrylaldehyde, 3-(5,5-dimethylcyclohex-1-en-1-yl)acrylaldehyde, 3-((1S,5R)-6,6-dimethylbicyclo[3.1.1]hept-2-en-3-yl)acrylaldehyde, (1R,2S,5R)-1-(3-(tert-butoxy)prop-1-en-1-yl)-2-isopropyl-5-methylcyclohexan-1-ol, 2-allyl-1-(3-hydroxyprop-1-en-1-yl)cyclohexan-1-ol, (3,3-dimethoxyprop-1-en-1-yl)benzene, are excluded.

Particularly, when X is of formula b), Z is a CH(OR^{a})(OR^{b}) group wherein R^{a} and R^{b} are identical and are a methyl group or a C₃₋₄ alkyl group, a C(═O)—R^{c} group wherein R^{c} is a C₁₋₄ alkyl group; or R^{a} and R^{b} are taken together and represent a C₃₋₆ alkanediyl group.

Particularly, when Z is a CH₂OH group, then X is of formula d) with the dotted line being a carbon-carbon single bond and p being 1.

Typical manners to execute the invention's process are reported herein below in the examples.

### EXAMPLES

The invention will now be described in further detail by way of the following examples, wherein the abbreviations have the usual meaning in the art, the temperatures are indicated in degrees centigrade (° C.). NMR spectra were acquired using either a Bruker Avance 11 Ultrashield 400 plus operating at 400 MHz, (¹H) and 100 MHz (¹³C) or a Bruker Avance III 500 operating at 500 MHz (¹H) and 125 MHz (¹³C) or a Bruker Avance III 600 cryoprobe operating at 600 MHz (¹H) and 150 MHz (¹³C). Spectra were internally referenced relative to tetramethyl silane 0.0 ppm. ¹H NMR signal shifts are expressed in δ ppm, coupling constants (J) are expressed in Hz with the following multiplicities: s, singlet; d, doublet; t, triplet; q, quartet; m, multiplet; b, broad (indicating unresolved couplings) and were interpreted using Bruker Topspin software. ¹³C NMR data are expressed in chemical shift S ppm and hybridization from DEPT 90 and DEPT 135 experiments, C, quaternary (s); CH, methine (d); CH₂, methylene (t); CH₃, methyl (q).

### Example 1

### Cross Metathesis Between 4,4-dimethyl-1-vinylcyclohexan-1-ol and 3,3-diethoxyprop-1-ene to Prepare (E)-1-(3,3-diethoxyprop-1-en-1-yl)-4,4-dimethylcyclohexan-1-ol

To a stirred solution of 2.5 g 4,4-dimethyl-1-vinylcyclohexan-1-ol (98% purity, 15.883 mmol) and 8.79 g 3,3-diethoxyprop-1-ene (63.533 mmol, 4 eq) in 10 mL EtOAc at 50° C. under Argon atmosphere was added 10 mg (0.0127 mmol, 0.080 mol %) GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6). Then 50 mg (0.0637 mmol, 0.40 mol %, GreenCat were dissolved in 5 mL of EtOAc and were added over a 3 hour period with a syringe pump. After the addition of 148 mg SnatchCat® (1,4-Bis(2-isocyanopropyl)piperazine, CAS 51641-96-4) the mixture was stirred one hour at room temperature and the solvent was evaporated under reduced pressure. The crude was purified by column chromatography (330 g cartridge, from Cyclo 9/AcOEt 1 to Cyclo 8/AcOEt 2) and 2.98 g (95% purity, 11.04 mmol, 69.5% yield) of (E)-1-(3,3-diethoxyprop-1-en-1-yl)-4,4-dimethylcyclohexan-1-ol and 2.9 g (25.4 mmol) of (E)-1,1,4,4-tetraethoxybut-2-ene were obtained. Unreacted 3,3-diethoxyprop-1-ene and 4,4-dimethyl-1-vinylcyclohexan-1-ol could be also isolated. Only very small quantities of (E)-1,1′-(ethene-1,2-diyl)bis(4,4-dimethylcyclohexan-1-ol) were observed in the GC of the crude (1.4%).

### (E)-1-(3,3-diethoxyprop-1-en-1-yl)-4,4-dimethylcyclohexan-1-ol

¹H-NMR (500.15 MHz, DMSO): 0.86 (s, 3H), 0.89 (s, 3H), 1.11 (t, 6H, J=7.0 Hz), 1.30-1.58 (m, 8H), 3.37-3.42 (m, 2H), 3.51-3.56 (m, 2H), 4.29 (br s OH, 1H), 4.84 (d, 1H, J=5.5 Hz), 5.54 (dd, 1H, J=15.8 Hz, J=5.6 Hz), 5.84 (d, 1H, J=15.8 Hz).

¹³C NMR (150 MHz, CDCl₃): δ 15.3, 25.2, 29.4, 31.2, 33.7, 34.6, 61.0, 71.0, 101.4, 124.8, 141.8.

### (E)-1,1,4,4-tetraethoxybut-2-ene

¹³C NMR (150 MHz, CDCl₃): δ 15.2, 61.1, 100.6, 131.2.

### (E)-1,1′-(ethene-1,2-diyl)bis(4,4-dimethylcyclohexan-1-ol)

¹³C NMR (150 MHz, CDCl₃): 525.5, 29.4, 30.8, 34.1, 34.8, 71.0, 135.1.

(E)-1-(3,3-diethoxyprop-1-en-1-yl)-4,4-dimethylcyclohexan-1-ol can be easily deprotected to (E)-3-(1-hydroxy-4,4-dimethylcyclohexyl)acrylaldehyde by the addition of a drop of H₃PO₄ and water at room temperature (quantitative yield):

98 mg (0.382 mmol) (E)-1-(3,3-diethoxyprop-1-en-1-yl)-4,4-dimethylcyclohexan-1-ol were dissolved in 2 mL of THF. Then 1 mL of water were added and 1 drop (2 mg, 5 mol %) of phosphoric acid (85%). The mixture was stirred for 10 min at room temperature and was diluted with 5 mL diethyl ether. After washing with 2 mL of a saturated aqueous NaHCO₃ solution the organic phase was dried over Na₂SO₄. The solvent was evaporated under reduced pressure and 68 mg (0.373 mmol, 98% yield) of pure (E)-3-(1-hydroxy-4,4-dimethylcyclohexyl)acrylaldehyde was obtained.

### (E)-3-(1-hydroxy-4,4-dimethylcyclohexyl)acrylaldehyde

¹H-NMR (500.15 MHz, CDCl₃): 0.94 (s, 1H), 0.98 (s, 1H), 1.25-1.75 (m, 8H), 1.82 (br s OH, 1H), 6.34 (dd, 1H, J=15.7 Hz, J=7.9 Hz), 6.88 (d, 1H, J=15.6 Hz), 9.58 (d, 1H, J=7.8 Hz).

¹³C NMR (150 MHz, CDCl₃): δ 15.3, 25.2, 29.3, 33.0, 34.0, 71.7, 129.3, 163.8, 193.9.

(E)-3-(1-hydroxy-4,4-dimethylcyclohexyl)acrylaldehyde could be transformed to the (E)-3-(4,4-dimethylcyclohex-1-en-1-yl)acrylaldehyde in the presence of a drop of H₃PO₄ and water at 100° C. (35 min):

To a stirred solution of 40 mg (0.2195 mmol) (E)-3-(1-hydroxy-4,4-dimethylcyclohexyl)acrylaldehyde in toluene was added phosphoric acid (85%) (one drop). The mixture was stirred at 100° C. for 35 min and diluted with 5 mL of diethyl ether. After washing with 2 mL of a saturated aqueous NaHCO₃ solution and with brine the organic phase was dried over Na₂SO₄. The solvent was evaporated under reduced pressure and 30 mg (0.1826 mmol, 83% yield) of pure (E)-3-(4,4-dimethylcyclohex-1-en-1-yl)acrylaldehyde were obtained.

### (E)-3-(4,4-dimethylcyclohex-1-en-1-yl)acrylaldehyde

¹H-NMR (500.15 MHz, CDCl₃): 0.94 (s, 6H), 1.49 (t, 2H, J=6.5 Hz), 2.04-2.07 (m, 2H), 2.17-2.23 (m, 2H), 6.09 (dd, 1H, J=15.7 Hz, J=8.1 Hz), 6.22-6.26 (m, 1H)), 7.10 (d, 1H, J=15.7 Hz), 9.56 (d, 1H, J=7.9 Hz).

¹³C NMR (150 MHz, CDCl₃): 522.0, 28.1, 28.9, 34.7, 40.6, 126.0, 134.3, 140.6, 156.0, 194.4.

### Example 2

### Cross Metathesis Between 1-vinylcyclohexan-1-ol and 3,3-diethoxyprop-1-ene to Prepare (E)-1-(3,3-diethoxyprop-1-en-1-yl)cyclohexan-1-ol

Preparation of (E)-1-(3,3-diethoxyprop-1-en-1-yl)cyclohexan-1-ol was performed according the procedure of Example 1 from 1-vinylcyclohexan-1-ol (86% yield).

### (E)-1-(3,3-diethoxyprop-1-en-1-yl)cyclohexan-1-ol

¹³C NMR (150 MHz, CDCl₃): δ 15.3, 22.0, 25.5, 37.7, 61.0, 71.2, 101.5, 124.8, 141.8.

(E)-1-(3,3-diethoxyprop-1-en-1-yl)cyclohexan-1-ol can be transformed to (E)-3-(1-hydroxycyclohexyl)acrylaldehyde and further to (E)-3-(cyclohex-1-en-1-yl)acrylaldehyde in the presence of an acid (pTsOH, oxalic acid, tartaric acid, KHSO₄) under Dean-Stark conditions (toluene, or cyclohexane).

### (E)-3-(1-hydroxycyclohexyl)acrylaldehyde

¹³C NMR (150 MHz, CDCl₃): δ 21.4, 25.1, 36.8, 71.9, 129.2, 163.7, 194.0.

### (E)-3-(cyclohex-1-en-1-yl)acrylaldehyde

¹³C NMR (150 MHz, CDCl₃): δ 21.9, 21.9, 24.3, 26.7, 125.8, 135.6, 141.3, 156.3, 194.4.

### Example 3

### Cross Metathesis Between 4,4-dimethyl-1-vinvlcyclohexan-1-ol and 2-vinyl-1,3-dioxolane to Prepare (E)-1-(2-(1,3-dioxolan-2-yl)vinyl)-4,4-dimethylcyclohexan-1-ol

To a stirred solution of 2.0 g 4,4-dimethyl-1-vinylcyclohexan-1-ol (96.3% purity, 12.486 mmol) and 3.75 g 2-vinyl-1,3-dioxolane (37.459 mmol, 3 eq) at 40° C. under Argon atmosphere was added 50.8 mg (0.0648 mmol, 0.5 mol %) GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6). After 20 minutes at 40° C. 25.4 mg (0.032 mmol, 0.26 mol %) GreenCat were added and the mixture was stirred at 400 for 20 min. After the addition of 107 mg SnatchCat® (1,4-Bis(2-isocyanopropyl)piperazine, CAS 51641-96-4) the mixture was stirred one hour at room temperature. The crude (5.29 g) was purified by column chromatography (330 g cartridge, from DCM 95/AcOEt 5 to DCM 90/AcOEt 10) and 2.26 g (9.99 mmol, 80.0% yield) of (E)-1-(2-(1,3-dioxolan-2-yl)vinyl)-4,4-dimethylcyclohexan-1-ol, 0.120 g (0.658 mmol, 5.3% yield) of (E)-3-(1-hydroxy-4,4-dimethylcyclohexyl)acrylaldehyde and 424 mg (2.45 mmol) of (E)-1,2-di(1,3-dioxolan-2-yl)ethene were obtained. Unreacted 2-vinyl-1,3-dioxolane (2.26 g, 22.5 mmol) and 4,4-dimethyl-1-vinylcyclohexan-1-ol (GC crude: 4.6%) could be also isolated. Only very small quantities of (E)-1,1′-(ethene-1,2-diyl)bis(4,4-dimethylcyclohexan-1-ol) were observed in the GC of the crude (1.7%, isolated 90 mg).

### (E)-1-(2-(1,3-dioxolan-2-yl)vinyl)-4,4-dimethylcyclohexan-1-ol

¹H-NMR (500.15 MHz, DMSO): 0.89 (s, 1H), 0.95 (s, 1H), 1.27-1.25 (m, 2H), 1.27 (br s OH, 1H), 1.48-1.68 (m, 10H), 3.87-3.94 (m, 2H), 3.98-4.05 (m, 2H), 5.26 (d, 1H, J=6.1 Hz), 5.73 (dd, 1H, J=15.7 Hz, J=6.3 Hz), 6.06 (d, 1H, J=15.7 Hz).

¹³C NMR (150 MHz, CDCl₃): 525.1, 29.4, 31.1, 33.5, 34.5, 65.0, 70.1, 103.8, 123.6, 144.2.

### (E)-3-(1-hydroxy-4,4-dimethylcyclohexyl)acrylaldehyde

¹³C NMR (150 MHz, CDCl₃): δ 15.3, 25.2, 29.3, 33.0, 34.0, 71.7, 129.3, 163.8, 193.9.

### (E)-1,2-di(1,3-dioxolan-2-yl)ethene

¹³C NMR (150 MHz, CDCl₃): δ 65.0, 102.3, 131.3.

### (E)-1,1′-(ethene-1,2-diyl)bis(4,4-dimethylcyclohexan-1-ol)

¹³C NMR (150 MHz, CDCl₃): δ 25.5, 29.4, 30.8, 34.1, 34.8, 71.0, 135.1.

(E)-1-(2-(1,3-dioxolan-2-yl)vinyl)-4,4-dimethylcyclohexan-1-ol can be transformed to (E)-2-(2-(4,4-dimethylcyclohex-1-en-1-yl)vinyl)-1,3-dioxolane in the presence of an acid (pTsOH, oxalic acid, tartaric acid, KHSO₄) under Dean-Stark conditions (toluene, or cyclohexane).

### (E)-2-(2-(4,4-dimethylcyclohex-1-en-1-yl)vinyl)-1,3-dioxolane

¹H-NMR (500.15 MHz, CDCl₃): 0.90 (s, 6H), 1.42 (t, 2H, J=6.5 Hz), 1.90-1.94 (m, 2H), 2.14-2.18 (m, 2H), 3.87-3.94 (m, 2H), 3.98-4.05 (m, 2H), 5.28 (d, 1H, J=6.4 Hz), 5.51 (dd, 1H, J=15.7 Hz, J=6.4 Hz), 5.75-5.77 (m, 1H), 6.41 (d, 1H, J=15.8 Hz).

¹³C NMR (125 MHz, CDCl₃): 522.1, 28.1, 28.9, 35.1, 39.9, 65.0, 104.5, 120.9, 131.3, 133.4, 138.6.

(E)-1-(2-(1,3-dioxolan-2-yl)vinyl)-4,4-dimethylcyclohexan-1-ol can be transformed to (E)-2-(2-(4,4-dimethylcyclohex-1-en-1-yl)vinyl)-1,3-dioxolane in the presence of POCl₃/Pyridine (0° C. to RT).

(E)-2-(2-(4,4-dimethylcyclohex-1-en-1-yl)vinyl)-1,3-dioxolane can be deprotected in quantitative yield to (E)-3-(4,4-dimethylcyclohex-1-en-1-yl)acrylaldehyde in the presence of water and H₃PO₄ (10 mol %) at room temperature or AcOH/water at 50° C. (30 min).

(E)-1-(2-(1,3-dioxolan-2-yl)vinyl)-4,4-dimethylcyclohexan-1-ol can be deprotected to (E)-3-(1-hydroxy-4,4-dimethylcyclohexyl)acrylaldehyde in the presence of water and H₃PO₄ (10 mol %) at room temperature or AcOH/water at 50° C. (30 min).

### Example 4

### Cross Metathesis Between 4,4-dimethyl-1-vinylcyclohexan-1-ol and allyl acetate to Prepare (E)-3-(1-hydroxy-4,4-dimethylcyclohexyl)allyl acetate

To a stirred solution of 4,4-dimethyl-1-vinylcyclohexan-1-ol (0.964 g, 6.25 mmol) and allyl acetate (1.95 g, 19.49 mmol) at 40° C. was added portion wise (6 times 0.25 mol % each 10 min) 1.25 mol % GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6) over 1 hour. After the last addition the mixture was cooled to room temperature and stirred over the week-end. Then 89.1 mg SnatchCat® (1,4-Bis(2-isocyanopropyl)piperazine, CAS 51641-96-4) were added and the mixture was stirred for 30 minutes at room temperature. The crude (2.35 g) was purified by column chromatography (80 g cartridge, from Cyclohexane 9/MTBE 1 to Cyclohexane 65/MTBE 35) and 0.590 g (2.61 mmol, 42% yield) of (E)-3-(1-hydroxy-4,4-dimethylcyclohexyl)allyl acetate were obtained. Unreacted 4,4-dimethyl-1-vinylcyclohexan-1-ol (546 mg, 3.53 mmol, 57% recycling yield) and (E)-but-2-ene-1,4-diyl diacetate (705 mg, and small amounts of (Z)-but-2-ene-1,4-diyl diacetate) could be also isolated. The formation of (E)-1,1′-(ethene-1,2-diyl)bis(4,4-dimethylcyclohexan-1-ol) was only observed in traces.

### (E)-3-(1-hydroxy-4,4-dimethylcyclohexyl)allyl acetate

¹H-NMR (500.15 MHz, CDCl₃): 0.90 (s, 3H), 0.95 (s, 3H), 1.20-1.27 (m, 2H), 1.33 (br s OH, 1H), 1.47-1.57 (m, 2H), 1.59-1.67 (m, 2H), 2.08 (s, 3H), 4.58 (dd, 2H, J=5.9 Hz, J=0.9 Hz), 5.80 (dt, 1H, J=15.6 Hz, J=6.0 Hz), 5.89 (dt, 1H, J=15.7 Hz, J=0.9 Hz).

¹³C NMR (150 MHz, CDCl₃): δ 21.0, 25.2, 29.4, 31.0, 33.7, 34.5, 64.7, 71.0, 121.5, 142.4, 170.8.

### Minor Isomer: (Z)-3-(1-hydroxy-4,4-dimethylcyclohexyl)allyl acetate (Characteristic Signals)

¹³C NMR (150 MHz, CDCl₃): δ 20.9, 64.2, 69.9, 126.7, 130.7, 170.8.

### (E)-but-2-ene-1,4-diyl diacetate

¹³C NMR (125 MHz, CDCl₃): δ 20.9, 63.9, 128.1, 170.7.

### (Z)-but-2-ene-1,4-diyl diacetate (Minor Isomer)

13C NMR (90 MHz, CDCl₃): δ 20.8, 60.0, 128.2, 170.5.

(E)-3-(1-hydroxy-4,4-dimethylcyclohexyl)allyl acetate (0.5 g, 2.209 mmol) can be deprotected to (E)-1-(3-hydroxyprop-1-en-1-yl)-4,4-dimethylcyclohexan-1-ol in the presence of KOH (372 mg, 6.62 mmol) at room temperature in 5 mL methanol (overnight). Methanol was evaporated under reduced pressure, water (2 mL) and MTBE (10 mL) were added. The mixture was stirred for 5 min and the phases were separated. The organic phase was washed twice with water (2 mL) and was dried over sodium sulfate. The solvent was evaporated under reduced pressure to give 0.399 g (2.165 mmol, 98% yield) of a white solid.

### (E)-1-(3-hydroxyprop-1-en-1-yl)-4,4-dimethylcyclohexan-1-ol

¹H-NMR (500.15 MHz, CDCl₃): 0.90 (s, 3H), 0.95 (s, 3H), 1.21-1.27 (m, 2H), 1.33 (br s OH, 1H), 1.48-1.56 (m, 4H), 1.60-1.67 (m, 2H), 4.17 (d, 2H, J=4.7 Hz), 5.84 (d, 1H, J=15.8 Hz), 5.88 (dt, 1H, J=15.7 Hz, J=4.7 Hz).

¹³C NMR (150 MHz, CDCl₃): δ 25.3, 29.4, 31.0, 33.9, 34.6, 63.3, 71.0, 126.5, 139.4.

### Example 5

### Cross Metathesis Between 4,4-dimethyl-1-vinylcyclohexan-1-ol and 1-(allyloxy)butane to Prepare (E)-1-(3-butoxyprop-1-en-1-yl)-4,4-dimethylcyclohexan-1-ol

To a stirred solution of 4,4-dimethyl-1-vinylcyclohexan-1-ol (2.889 mg, 18.73 mmol) and 1-(allyloxy)butane (6.42 g, 56.2 mmol) at 40° C. was added portion wise (3 times 0.25 mol % each 25 min) 0.75 mol % GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6) over 1 h 15 min. The mixture was cooled to room temperature and stirred overnight. Then 154 mg SnatchCat® (1,4-Bis(2-isocyanopropyl)piperazine, CAS 51641-96-4) were added and the mixture was stirred for 30 minutes at room temperature. The crude was purified by column chromatography (330 g cartridge, from Cyclohexane 95/MTBE 5 to Cyclohexane 7/MTBE 3) and 1.77 g (7.36 mmol, 39% yield) of (E)-1-(3-butoxyprop-1-en-1-yl)-4,4-dimethylcyclohexan-1-ol were obtained. Unreacted 1-(allyloxy)butane (2.82 g, 24.7 mmol), 4,4-dimethyl-1-vinylcyclohexan-1-ol (1.53 g, 9.95 mmol, 53% recycling yield) and (E)-1,4-dibutoxybut-2-ene (2.31 g, 11.54 mmol) could be also isolated. The formation of (E)-1,1′-(ethene-1,2-diyl)bis(4,4-dimethylcyclohexan-1-ol) was only observed in traces.

### (E)-1-(3-butoxyprop-1-en-1-yl)-4,4-dimethylcyclohexan-1-ol

¹H-NMR (500.15 MHz, CDCl₃): 0.89 (s, 3H), 0.91 (t, 3H, J=4.7 Hz), 0.95 (s, 3H), 1.19-1.26 (m, 2H), 1.34-1.41 (m, 2H), 1.49-1.60 (m, 6H), 1.61-1.68 (m, 2H), 3.42 (t, 2H, J=6.7 Hz), 3.97 (d, 2H, J=5.0 Hz), 5.79 (dt, 1H, J=15.7 Hz, J=5.3 Hz), 5.84 (d, 1H, J=15.8 Hz).

¹³C NMR (150 MHz, CDCl₃): δ 13.9, 19.4, 25.4, 29.4, 31.0, 31.8, 33.8, 34.7, 70.2, 71.0, 71.1, 124.2, 140.5.

### (E)-1,4-dibutoxybut-2-ene

¹³C NMR (150 MHz, CDCl₃): 513.9, 19.4, 31.9, 70.2, 70.8, 129.5.

### Example 6

### Cross Metathesis Between 4,4-dimethyl-1-vinylcyclohexan-1-ol and 1-(1-(allyloxy)ethoxy)butane to Prepare (E)-1-(3-(1-butoxyethoxy)prop-1-en-1-yl)-4,4-dimethylcyclohexan-1-ol

To a stirred solution of 4,4-dimethyl-1-vinylcyclohexan-1-ol (0.576 g, 3.73 mmol) and 1-(1-(allyloxy)ethoxy)butane 0.59 g, 3.73 mmol) at 40° C. was added portion wise (3 times 0.5 mol % each 30 min) 1.5 mol % GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6) over 1 h 30 min. The mixture was cooled to room temperature and stirred overnight. Then 64.2 mg SnatchCat® (1,4-Bis(2-isocyanopropyl)piperazine, CAS 51641-96-4) were added and the mixture was stirred for 30 minutes at room temperature. The crude was purified by column chromatography (330 g cartridge, from Cyclohexane 99/MTBE 1 to Cyclohexane 7/MTBE 3) and 0.408 g (1.43 mmol, 38% yield) of (E)-1-(3-(1-butoxyethoxy)prop-1-en-1-yl)-4,4-dimethylcyclohexan-1-ol were obtained. Unreacted 1-(1-(allyloxy)ethoxy)butane, 4,4-dimethyl-1-vinylcyclohexan-1-ol (180 mg, 1.17 mmol, 31% recycling yield) and (E)-6,13-dimethyl-5,7,12,14-tetraoxaoctadec-9-ene (18 mg, 0.06 mmol) could be also isolated. The formation of (E)-1,1′-(ethene-1,2-diyl)bis(4,4-dimethylcyclohexan-1-ol) was only observed in traces.

### (E)-1-(3-(1-butoxyethoxy)prop-1-en-1-yl)-4,4-dimethylcyclohexan-1-ol

¹H-NMR (500.15 MHz, CDCl₃): 0.89 (s, 3H), 0.93 (t, 3H, J=4.7 Hz), 0.95 (s, 3H), 1.20-1.27 (m, 1H), 1.32 (d, J=5.4 Hz), 1.34-1.44 (m, 2H), 1.50-1.59 (m, 6H), 1.60-1.68 (m, 2H), 3.43 (dt, 1H, J=9.3 Hz, J=6.7 Hz), 3.57 (dt, 1H, J=9.4 Hz, J=6.7 Hz), 4.00 (dd, 1H, J=12.4 Hz, J=5.3 Hz), 4.12 (dd, 1H, J=12.3 Hz, J=5.2 Hz), 4.73 (q, 1H, J=5.4 Hz), 5.79 (dt, 1H, J=15.6 Hz, J=5.4 Hz), 5.85 (d, 1H, J=15.8 Hz).

¹³C NMR (150 MHz, CDCl₃): δ 13.9, 19.4, 19.8, 25.4, 29.4, 31.0, 32.0, 33.8, 34.7, 65.0, 65.4, 71.0, 99.2, 124.0, 140.5.

### (E)-6,13-dimethyl-5,7,12,14-tetraoxaoctadec-9-ene

¹³C NMR (150 MHz, CDCl₃): δ 13.9, 19.4, 19.8, 32.0, 64.9, 64.9, 65.1, 65.1, 99.2, 129.2.

### Example 7

### Cross Metathesis Between 4,4-dimethyl-1-vinylcyclohexan-1-ol and prop-2-ene-1,1-diyl diacetate to Prepare (E)-3-(1-hydroxy-4,4-dimethylcyclohexyl)prop-2-ene-1,1-diyl diacetate

To a stirred solution of 2.5 g 4,4-dimethyl-1-vinylcyclohexan-1-ol (98% purity, 15.883 mmol) and 10.05 g prop-2-ene-1,1-diyl diacetate (63.53 mmol, 4 eq) in 10 mL EtOAc at 50° C. under Argon atmosphere was added 20 mg (0.0254 mmol, 0.160 mol %) GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6). Then 110 mg (0.0637 mmol, 0.88 mol %, GreenCat were dissolved in 10 mL of EtOAc and were added over a 3 hour period with a syringe pump. After the addition of 298 mg SnatchCat® (1,4-Bis(2-isocyanopropyl)piperazine, CAS 51641-96-4) the mixture was stirred one hour at room temperature and the solvent was evaporated under reduced pressure. The crude was purified by column chromatography (330 g cartridge, from Cyclo 9/AcOEt 1 to Cyclo 8/AcOEt 2) and 2.85 g (99% purity, 9.92 mmol, 62.4% yield) of (E)-3-(1-hydroxy-4,4-dimethylcyclohexyl)prop-2-ene-1,1-diyl diacetate were obtained. Unreacted prop-2-ene-1,1-diyl diacetate (8.4 g, 0.0536 mmol) and 4,4-dimethyl-1-vinylcyclohexan-1-ol could be also isolated. Only very small quantities of (E)-1,1′-(ethene-1,2-diyl)bis(4,4-dimethylcyclohexan-1-ol) were observed in the GC of the crude (<1%). The formation of (E)-but-2-ene-1,1,4,4-tetrayl tetraacetate was not observed.

### (E)-3-(1-hydroxy-4,4-dimethylcyclohexyl)prop-2-ene-1,1-diyl diacetate

¹H-NMR (500.15 MHz, CDCl₃): 0.86 (s, 3H), 0.89 (s, 3H), 1.20-1.27 (m, 2H), 1.39 (br s OH, 1H), 1.46-1.57 (m, 4H), 1.59-1.68 (m, 2H), 2.09 (s, 6H), 5.79 (dd, 1H, J=15.7 Hz, J=6.2 Hz), 6.12 (d, 1H, J=15.7 Hz), 7.15 (d, 1H, J=6.1 Hz).

¹³C NMR (150 MHz, CDCl₃): δ 20.9, 25.0, 29.4, 31.2, 33.5, 34.3, 71.0, 89.4, 120.7, 144.4, 168.7.

Transformation of (E)-3-(1-hydroxy-4,4-dimethylcyclohexyl)prop-2-ene-1,1-diyl diacetate to (E)-3-(4,4-dimethylcyclohex-1-en-1-yl)acrylaldehyde was performed in the presence of potassium bisulfate and water (in toluene, 110° C., 1 hour).

Transformation of (E)-3-(1-hydroxy-4,4-dimethylcyclohexyl)prop-2-ene-1,1-diyl diacetate to the (E)-3-(4,4-dimethylcyclohex-1-en-1-yl)prop-2-ene-1,1-diyl diacetate was performed if the presence of POCl₃/Pyridine (0° C.->RT, 16 h).

### (E)-3-(4,4-dimethylcyclohex-1-en-1-yl)prop-2-ene-1,1-diyl diacetate

¹³C NMR (150 MHz, CDCl₃): δ 20.9, 22.0, 28.1, 28.9, 35.0, 39.9, 90.4, 117.8, 132.9, 133.0, 138.9, 168.8.

Preparation of (E)-3-(1-hydroxycyclohexyl)prop-2-ene-1,1-diyl diacetate: according the previous procedure from 1-vinylcyclohexan-1-ol (70% yield).

¹³C NMR (150 MHz, CDCl₃): 520.9, 25.0, 29.4, 31.2, 33.5, 34.3, 71.0, 89.4, 120.7, 144.4, 168.7.

Transformation of (E)-3-(1-hydroxycyclohexyl)prop-2-ene-1,1-diyl diacetate to the (E)-3-(cyclohex-1-en-1-yl)prop-2-ene-1,1-diyl diacetate was performed if the presence of POCl₃/Pyridine (0° C.->RT, 16 h).

### (E)-3-(cyclohex-1-en-1-yl)prop-2-ene-1,1-diyl diacetate

¹³C NMR (150 MHz, CDCl₃): 520.9, 22.2, 22.2, 24.2, 26.0, 90.4, 117.7, 133.8, 134.2, 139.3, 168.7.

(E)-3-(cyclohex-1-en-1-yl)prop-2-ene-1,1-diyl diacetate can be deprotected to (E)-3-(cyclohex-1-en-1-yl)acrylaldehyde in the presence of 1 eq triethylamine in MeOH (4 hours at room temperature).

### (E)-3-(cyclohex-1-en-1-yl)acrylaldehyde

¹³C NMR (150 MHz, CDCl₃): δ 21.9, 21.9, 24.3, 26.7, 125.8, 135.6, 141.3, 156.3, 194.4.

### Example 8

### Cross Metathesis Between 4,4-dimethyl-1-vinylcyclohex-1-ene and prop-2-ene-1,1-diyl diacetate to Prepare (E)-3-(4,4-dimethylcyclohex-1-en-1-yl)prop-2-ene-1,1-diyl diacetate and (E)-3-(4,4-dimethylcyclohex-1-en-1-yl)acrylaldehyde

To a stirred solution of 0.56 g 4,4-dimethyl-1-vinylcyclohex-1-ene (92.8% purity, 3.81 mmol) and 2.57 g prop-2-ene-1,1-diyl diacetate (15.26 mmol, 4 eq) in 7.4 mL EtOAc at room temperature under Argon atmosphere was added 299 mg (0.381 mmol, 10 mol %) GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6) in small portions (1 mol %, 2 mol %, 2 mol % and 5 mol %). After every addition the mixture was heated for at 50° C. for 1-3 hours. After the addition of 472 mg (2.29 mmol) SnatchCat® (1,4-Bis(2-isocyanopropyl)piperazine, CAS 51641-96-4) the mixture was stirred one hour at room temperature. The solvent was evaporated under reduced pressure. The crude was purified by column filtration through a pad of silica and the volatiles (unreacted prop-2-ene-1,1-diyl diacetate) were distilled of by a Kugelrohr distillation. 779 mg (2.778 mmol, 73% yield) of (E)-3-(4,4-dimethylcyclohex-1-en-1-yl)prop-2-ene-1,1-diyl diacetate and 98 mg of (E)-but-2-ene-1,1,4,4-tetrayl tetraacetate (0.34 mmol) were obtained. Unreacted 4,4-dimethyl-1-vinylcyclohex-1-ene could be also isolated. Only small quantities of (E)-1,2-bis(4,4-dimethylcyclohex-1-en-1-yl)ethene were isolated (57 mg, 0.234 mmol).

### (E)-3-(4,4-dimethylcyclohex-1-en-1-yl)prop-2-ene-1,1-diyl diacetate

¹H-NMR (500.15 MHz, CDCl₃): 0.91 (s, 6H), 1.43 (t, 2H, J=6.4 Hz), 1.92-1.95 (m, 2H), 2.09 (s, 6H), 2.11-2.15 (m, 2H), 5.54 (dd, 1H, J=15.8 Hz, J=6.7 Hz), 5.81-5.84 (m, 1H), 6.50 (d, 1H, J=15.8 Hz), 7.18 (d, 1H, J=6.7 Hz).

¹³C NMR (150 MHz, CDCl₃): 920.9, 22.0, 28.1, 28.9, 35.0, 39.9, 90.4, 117.8, 132.9, 133.0, 138.9, 168.8.

(E)-3-(4,4-dimethylcyclohex-1-en-1-yl)prop-2-ene-1,1-diyl diacetate can be deprotected to (E)-3-(4,4-dimethylcyclohex-1-en-1-yl)acrylaldehyde in the presence of 1 eq triethylamine in MeOH (4 hours at room temperature).

### (E)-3-(4,4-dimethylcyclohex-1-en-1-yl)acrylaldehyde

¹³C NMR (150 MHz, CDCl₃): 522.0, 28.1, 28.9, 34.7, 40.6, 126.0, 134.3, 140.6, 156.0, 194.4.

### (E)-but-2-ene-1,1,4,4-tetrayl tetraacetate

¹³C NMR (150 MHz, CDCl₃): δ 20.8, 87.5, 128.8, 168.5.

### Example 9

### Cross Metathesis Between 4,4-dimethyl-1-vinylcyclohex-1-ene and 3,3-diethoxyprop-1-ene to Prepare (E)-1-(3,3-diethoxyprop-1-en-1-yl)-4,4-dimethylcyclohex-1-ene and (E)-3-(4,4-dimethylcyclohex-1-en-1-yl)acrylaldehyde

To a stirred solution of 0.500 g 4,4-dimethyl-1-vinylcyclohex-1-ene (92.8% purity, 3.406 mmol) and 1.33 g 3,3-diethoxyprop-1-ene (10.22 mmol, 3 eq) in 6 mL EtOAc at 50° C. under Argon atmosphere was added 26.7 mg (0.0341 mmol, 1 mol %) GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6). The mixture was heated for 30 minutes at 50° C. Then four times 26.7 mg (0.0341 mmol, 1 mol %) GreenCat were added every 30 minutes and the mixture was stirred further 30 min at 50° C. After the addition of 179 mg (0.86 mmol) SnatchCat® (1,4-Bis(2-isocyanopropyl)piperazine, CAS 51641-96-4) the mixture was stirred one hour at room temperature. The solvent was evaporated under reduced pressure. The crude was purified by column filtration (25 g cartridge, from Cyclo 95/AcOEt 5 to Cyclo 9/AcOEt 1) and 0.748 g (2.96 mmol, 87% yield) of (E)-1-(3,3-diethoxyprop-1-en-1-yl)-4,4-dimethylcyclohex-1-ene and 374 mg (1.61 mmol) of (E)-1,1,4,4-tetraethoxybut-2-ene were obtained. Unreacted 3,3-diethoxyprop-1-ene and 4,4-dimethyl-1-vinylcyclohex-1-ene (GC 9%) could be also isolated. Only very small quantities of E)-1,2-bis(4,4-dimethylcyclohex-1-en-1-yl)ethene were observed in the GC of the crude (2.2%).

### (E)-1-(3,3-diethoxyprop-1-en-1-yl)-4,4-dimethylcyclohex-1-ene

¹H-NMR (500.15 MHz, DMSO): 0.88 (s, 6H), 1.10 (t, 4H, J=7.1 Hz), 1.38 (t, 2H, J=6.6 Hz), 1.88-1.91 (m, 2H), 2.04-2.09 (m, 2H), 3.39-3.47 (m, 2H), 3.50-3.57 (m, 2H), 4.90 (d, 1H, J=5.6 Hz), 5.45 (dd, 1H, J=16.0 Hz, J=5.5 Hz), 5.72-5.75 (m, 1H), 6.27 (d, 1H, J=16.0 Hz).

¹³C NMR (125 MHz, CDCl₃): δ 15.3, 22.2, 28.2, 28.9, 35.2, 39.9, 61.0, 102.3, 122.6, 130.3, 133.6, 136.3.

(E)-1-(3,3-diethoxyprop-1-en-1-yl)-4,4-dimethylcyclohex-1-ene can be deprotected to (E)-3-(4,4-dimethylcyclohex-1-en-1-yl)acrylaldehyde in the presence of AcOH and water at room temperature (quantitative yield).

### (E)-3-(4,4-dimethylcyclohex-1-en-1-yl)acrylaldehyde

¹³C NMR (150 MHz, CDCl₃): 522.0, 28.1, 28.9, 34.7, 40.6, 126.0, 134.3, 140.6, 156.0, 194.4.

### Example 10

### Cross Metathesis Between 4,4-dimethyl-1-vinylcyclohex-1-ene and allyl acetate to Prepare (E)-3-(4,4-dimethylcyclohex-1-en-1-yl)allyl acetate and (E)-3-(4,4-dimethylcyclohex-1-en-1-yl)prop-2-en-1-ol

To a stirred solution of 0.500 g 4,4-dimethyl-1-vinylcyclohex-1-ene (91.9% purity, 3.37 mmol) and 1.10 g allyl acetate (11.01 mmol) in 6 mL EtOAc at room temperature under Argon atmosphere was added 28.8 mg (0.037 mmol, 1 mol %) GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6). The mixture was stirred for 20 minutes at room temperature. Then 2 times 28.8 mg (0.037 mmol, 1 mol %) GreenCat were added every 30 minutes and the mixture was stirred further 30 min at room temperature. After the addition of 151 mg (0.73 mmol) SnatchCat® (1,4-Bis(2-isocyanopropyl)piperazine, CAS 51641-96-4) the mixture was stirred one hour at room temperature. The solvent was evaporated under reduced pressure. The crude (1.76 g) was purified by column filtration (25 g cartridge, from Cyclo 98/MTBE 2 to Cyclo 75/MTBE 25) and 0.491 g (2.36 mmol, 70% yield) of (E)-3-(4,4-dimethylcyclohex-1-en-1-yl)allyl acetate and 368 mg (2.13 mmol) of (E)-but-2-ene-1,4-diyl diacetate were obtained. Unreacted allyl acetate and 4,4-dimethyl-1-vinylcyclohex-1-ene (GC 27%) could be also isolated. Only very small quantities of E)-1,2-bis(4,4-dimethylcyclohex-1-en-1-yl)ethene were observed in the GC of the crude (1.0%).

### (E)-3-(4,4-dimethylcyclohex-1-en-1-yl)allyl acetate

¹H-NMR (500.15 MHz, CDCl₃): 0.91 (s, 6H), 1.42 (t, 2H, J=6.4 Hz), 1.90-1.93 (m, 2H), 2.07 (s, 3H), 2.12-2.17 (m, 2H), 4.60 (d, 2H, J=6.8 Hz), 5.62 (dt, 1H, J=15.7 Hz, J=6.6 Hz), 5.71-5.84 (m, 1H), 6.29 (d, 1H, J=15.7 Hz).

¹³C NMR (125 MHz, CDCl₃): δ 21.1, 22.2, 28.1, 28.9, 35.1, 39.9, 65.6, 119.0, 130.3, 133.5, 138.0, 171.0.

(E)-3-(4,4-dimethylcyclohex-1-en-1-yl)allyl acetate can be deprotected to (E)-3-(4,4-dimethylcyclohex-1-en-1-yl)prop-2-en-1-ol in quantitative yield (KOH, MeOH, room temperature, 15 h).

### (E)-3-(4,4-dimethylcyclohex-1-en-1-yl)prop-2-en-1-ol

¹H-NMR (500.15 MHz, CDCl₃): 0.91 (s, 6H), 1.43 (t, 2H, J=6.5 Hz), 1.55 (br s OH, 1H), 1.90-1.93 (m, 2H), 2.12-2.17 (m, 2H), 4.19 (d, 2H, J=6.8 Hz), 5.67-5.70 (m, 1H), 5.71 (dt, 1H, J=15.7 Hz, J=6.4 Hz), 6.24 (d, 1H, J=15.7 Hz).

¹³C NMR (125 MHz, CDCl₃): δ 22.3, 28.2, 28.9, 35.2, 39.8, 64.0, 124.4, 129.2, 133.7, 135.0.

(E)-1-(3-butoxyprop-1-en-1-yl)-4,4-dimethylcyclohex-1-ene could be prepared from 4,4-dimethyl-1-vinylcyclohex-1-ene and 3 eq 1-(allyloxy)butane by using the previous cross metathesis protocol (29% conversion after 1 h 30 min using 2 mol % Green Cat).

### (E)-1-(3-butoxyprop-1-en-1-yl)-4,4-dimethylcyclohex-1-ene

¹H-NMR (500.15 MHz, CDCl₃): 0.90 (s, 6H), 0.91 (t, 3H, J=7.4 Hz), 1.34-1.40 (m, 2H), 1.41 (t, 2H, J=6.5 Hz), 1.54-1.59 (m, 2H), 1.88-1.92 (m, 2H), 2.12-2.17 (m, 2H), 3.42 (t, 2H, J=6.7 Hz), 4.00 (d, 2H, J=6.4 Hz), 5.64 (dt, 1H, J=15.7 Hz, J=6.4 Hz), 5.64-5.67 (m, 1H), 6.23 (d, 1H, J=15.7 Hz).

¹³C NMR (150 MHz, CDCl₃): δ 14.0, 19.4, 22.3, 28.2, 28.9, 31.9, 35.3, 39.8, 70.1, 71.8, 122.2, 128.9, 133.9, 136.0.

### Example 11

### Cross Metathesis Between (E)-1-methyl-4-(2-methylbuta-1,3-dien-1-yl)benzene and 3,3-diethoxyprop-1-ene to Prepare 1-((1E,3E)-5,5-diethoxy-2-methylpenta-1,3-dien-1-yl)-4-methylbenzene and (2E,4E)-4-methyl-5-(p-tolyl)penta-2,4-dienal

To a stirred solution of 0.55 g (E)-1-methyl-4-(2-methylbuta-1,3-dien-1-yl)benzene prepared via a Wittig reaction from commercial (E)-2-methyl-3-(p-tolyl)acrylaldehyde (CAS 93614-82-5) according to a literature procedure (K. P. S. Cheung, D. Kurandina, T. Yata, and V. Gevorgyan J. Am. Chem. Soc. 2020, 142, 9932-9937) (99% purity, 3.476 mmol) and 1.444 g 3,3-diethoxyprop-1-ene (10.427 mmol, 3 eq) in 10 mL EtOAc at 50° C. under Argon atmosphere was added 10 mg (0.0127 mmol, 0.36 mol %) GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6). The mixture was stirred for 30 minutes at 50° C. Then three times 10 mg (0.0127 mmol, 0.36 mol %) GreenCat were added every 30 minutes and the mixture was stirred further 30 min. After the addition of 100 mg SnatchCat® (1,4-Bis(2-isocyanopropyl)piperazine, CAS 51641-96-4) and 30 minutes of stirring the solvent was evaporated under reduced pressure. The crude was purified by column chromatography (80 g cartridge, from Cyclo 97.5/AcOEt 2.5 to Cyclo 9/AcOEt 1) and 405 mg (2.022 mmol, 58.2% yield) of (2E,4E)-4-methyl-5-(p-tolyl)penta-2,4-dienal and 63 mg (0.231 mmol, 6.6% yield) of 1-((1E,3E)-5,5-diethoxy-2-methylpenta-1,3-dien-1-yl)-4-methylbenzene were obtained. Unreacted 3,3-diethoxyprop-1-ene and (E)-1-methyl-4-(2-methylbuta-1,3-dien-1-yl)benzene (177 mg, 1.118 mmol, 32% recycled yield) could be also isolated. The formation of 4,4′-((1E,3E,5E)-2,5-dimethylhexa-1,3,5-triene-1,6-diyl)bis(methylbenzene) was not observed.

### 1-((1E,3E)-5,5-diethoxy-2-methylpenta-1,3-dien-1-yl)-4-methylbenzene

¹³C NMR (100 MHz, CDCl₃): 5101.95, 103.34 (characteristic signals).

### (2E,4E)-4-methyl-5-(p-tolyl)penta-2,4-dienal

¹H-NMR (500.15 MHz, CDCl₃): 2.10 (s, 3H), 2.38 (s, 3H), 6.26 (dd, 1H, J=15.4 Hz, J=7.7 Hz), 6.92 (br s, 1H)), 7.28 (d, 1H, J=15.4 Hz), 7.21 (d, 2H, J=8.0 Hz), 7.30 (d, 2H, J=8.4 Hz), 9.64 (d, 1H, J=7.8 Hz).

¹³C NMR (100 MHz, CDCl₃): δ 13.9, 21.3, 127.8, 129.2, 129.7, 133.4, 133.7, 138.4, 141.0, 158.2, 193.9.

1-((1E,3E)-5,5-diethoxy-2-methylpenta-1,3-dien-1-yl)-4-methylbenzene could be transformed to (2E,4E)-4-methyl-5-(p-tolyl)penta-2,4-dienal in the presence of AcOH/water at room temperature (quantitative yield).

### Example 12

### Cross Metathesis Between (E)-1-methyl-4-(2-methylbuta-1,3-dien-1-yl)benzene and 3,3-diethoxyprop-1-ene to Prepare (2E,4E)-4-methyl-5-(p-tolyl)penta-2,4-diene-1,1-diyl diacetate and (2E,4E)-4-methyl-5-(p-tolyl)penta-2,4-dienal

To a stirred solution of 0.50 g (E)-1-methyl-4-(2-methylbuta-1,3-dien-1-yl)benzene (99% purity, 3.160 mmol) and 1.499 g prop-2-ene-1,1-diyl diacetate 9.48 mmol, 3 eq) in 20 mL dichloromethane at room temperature under Argon atmosphere was added 25 mg (0.0316 mmol, 1 mol %) GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6). The mixture was stirred for 30 minutes at room temperature. Then 125 mg (0.158 mmol, 5 mol %) GreenCat were added and the mixture was stirred over night at room temperature. After the addition of 12.5 mg (0.0158 mmol, 0.5 mol %) GreenCat the mixture was refluxed for 5 hours. After the addition of 243 mg SnatchCat® (1,4-Bis(2-isocyanopropyl)piperazine, CAS 51641-96-4) and 30 minutes of stirring the solvent was evaporated under reduced pressure. The crude was purified by column chromatography (80 g cartridge, from Cyclo 95/AcOEt 5 to Cyclo 9/AcOEt 1) and 0.766 g of a mixture of (2E,4E)-4-methyl-5-(p-tolyl)penta-2,4-diene-1,1-diyl diacetate and (2E,4E)-4-methyl-5-(p-tolyl)penta-2,4-dienal were obtained (containing some amounts of prop-2-ene-1,1-diyl diacetate). (2E,4E)-4-methyl-5-(p-tolyl)penta-2,4-diene-1,1-diyl diacetate could be deprotected to (2E,4E)-4-methyl-5-(p-tolyl)penta-2,4-dienal in the presence of 1 eq triethylamine in MeOH (4 hours at room temperature).

### (2E,4E)-4-methyl-5-(p-tolyl)penta-2,4-diene-1,1-diyl diacetate

¹H-NMR (500.15 MHz, CDCl₃): 1.99 (s, 3H), 2.11 (s, 6H), 2.34 (s, 3H), 5.73 (dd, 1H, J=15.4 Hz, J=6.6 Hz), 6.61 (br s, 1H)), 6.68 (d, 1H, J=15.7 Hz). (characteristic signals from a mixture with (2E,4E)-4-methyl-5-(p-tolyl)penta-2,4-dienal).

¹³C NMR (100 MHz, CDCl₃): 913.7, 20.9, 90.1, 120.5, 129.0, 133.2, 134.2, 134.9, 137.0, 141.0, 168.8. (characteristic signals from a mixture with (2E,4E)-4-methyl-5-(p-tolyl)penta-2,4-dienal).

### (2E,4E)-4-methyl-5-(p-tolyl)penta-2,4-dienal

¹³C NMR (100 MHz, CDCl₃): δ 13.9, 21.3, 127.8, 129.2, 129.7, 133.4, 133.7, 138.4, 141.0, 158.2, 193.9.

### Example 13

### Preparation of 2-methyl-1-(p-tolyl)but-3-en-1-ol from 4-methylbenzaldehyde

To a cooled solution (0° C.) of 494.4 mL 1-methyl-2-propenylmagnesium chloride (0.5 M in THF, 247.2 mmol, 1.1 eq) was added slowly a solution of 4-methylbenzaldehyde (27.0 g, 224.7 mmol) in 135 mL THF. The internal temperature did not exceed 5° C. during the addition of the aldehyde. The mixture was further stirred at 0° C. overnight (16 hours) and analysed by GC. The reaction mixture was added slowly to a cooled solution of 16.2 g AcOH (269.7 mmol) in 200 ml water. The phases were separated and the aqueous phase was extracted twice with 150 mL TBME. The combined organic phase were washed with a saturated aqueous NaHCO₃ solution and a saturated aqueous NaCl solution. After drying over Na₂SO₄ the solvent was evaporated under reduced pressure (500-4 mbar, 50° C.). The crude (44.3 g) was purified by a distillation through a Vigreux column under reduced pressure (oil bath 120° C., 900-3 mbar, bp 90° C./3 mbar). 39.0 g (221.3 mmol, 98.4% yield) 2-methyl-1-(p-tolyl)but-3-en-1-ol (syn/anti mixture) of a colourless liquid were obtained.

NMR analysis results in CDCl₃ were in accordance with data from literature for the syn isomer:
Shibata, I.; Yoshimura, N.; Yabu, M. Baba, A. Eur. J. Org. Chem. 2001, 3207-3211.
S. Hayashi, K. Hirano, H. Yorimitsu, K. Oshima Org. Lett. 2005, 7, 16, 3577-3579.

### 2-methyl-1-(p-tolyl)but-3-en-1-ol (Syn Isomer)

¹³C NMR (125 MHz, CDCl₃): 514.2, 21.1, 44.6, 77.2, 115.4, 126.5, 128.8, 137.0, 139.6, 140.4.

### 2-methyl-1-(p-tolyl)but-3-en-1-ol (Anti Isomer)

¹³C NMR (125 MHz, CDCl₃): 516.6, 21.1, 46.2, 77.7, 116.7, 126.8, 128.9, 137.3, 139.5, 140.8.

### Example 14

### Preparation of 2-methyl-1-(p-tolyl)but-3-en-1-yl acetate from 2-methyl-1-(p-tolyl)but-3-en-1-ol

To 9.94 g (56.397 mmol) 2-methyl-1-(p-tolyl)but-3-en-1-ol was added DMAP (172 mg, 1.41 mmol, 2.5 mol %) and triethylamine (5.71 g, 56.4 mmol, 1 eq) under stirring and N₂ atmosphere. Then Acetic anhydride (11.515 g, 112.79 mmol, 2 eq) was added slowly (exothermic). The mixture was stirred 1.5 hours at room temperature (complete conversion). The mixture was cooled with an ice bath (0° C.) and 5 mL of water were added slowly (hydrolysis of residual Ac₂O). 13.8 g of an aqueous 25% NaOH solution (1.5 eq) were added slowly. After stirring for 30 min, 25 mL of MTBE were added. The phases were separated. The aqueous phase was extracted once with 25 mL MTBE. The combined organic phases were washed twice with water (15 mL), and then once with a saturated aqueous NaHCO₃ solution (15 mL) and once with water (15 mL). After a final wash with brine (10 mL) the organic phase was dried over sodium sulfate, filtered and evaporated under reduced pressure (50° C., 50 mbar). The crude (12.3 g) was purified by flash chromatography (220 g cartridge, eluent from pentane 100% to pentane 95/MTBE 5). 2-methyl-1-(p-tolyl)but-3-en-2-yl acetate (1/1 mixture of diastereomers) was isolated (11.93 g, 54.64 mmol, 97 yield) as a colourless liquid.

¹³C NMR (125 MHz, CDCl₃): 516.4, 21.2, 21.2, 43.5, 78.9, 115.5, 127.2, 128.9, 136.1, 137.6, 140.0, 170.2.

¹³C NMR (125 MHz, CDCl₃): 515.4, 21.1, 21.1, 42.7, 78.9, 115.5, 127.1, 128.8, 136.0, 137.4, 139.2, 170.2.

### Example 15

### Cross Metathesis Between 2-methyl-1-(p-tolyl)but-3-en-1-ol and 3,3-diethoxyprop-1-ene to Prepare (E)-5,5-diethoxy-2-methyl-1-(p-tolyl)pent-3-en-1-ol

To a stirred solution of 2-methyl-1-(p-tolyl)but-3-en-1-ol (1.0 g, 5.674 mmol) and 3,3-diethoxyprop-1-ene (2.2 g, 17.02 mmol) in EtOAc (10 ml) at room temperature was added portion wise (5 times 1.6 mol %) 8 mol % GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6). After 67 hours reaction time at room temperature 650 mg SnatchCat® (1,4-Bis(2-isocyanopropyl)piperazine, CAS 51641-96-4) were added and the mixture was stirred for 30 minutes at room temperature. The solvent was evaporated under reduced pressure (20 mbar, 50° C.). The crude was purified by column chromatography (40 g cartridge, from Cyclohexane 98/MTBE 2 to Cyclohexane 9/AcOEt 1) and 0.330 g (1.18 mmol, 21% yield) of (E)-5,5-diethoxy-2-methyl-1-(p-tolyl)pent-3-en-1-ol and were obtained. Unreacted 3,3-diethoxyprop-1-ene, 2-methyl-1-(p-tolyl)but-3-en-1-ol (369 mg, 2.10 mmol, 37% recycling yield) and (E)-1,1,4,4-tetraethoxybut-2-ene could be also isolated. Only small amounts of (E)-2,5-dimethyl-1,6-di-p-tolylhex-3-ene-1,6-diol were formed.

### (E)-5,5-diethoxy-2-methyl-1-(p-tolyl)pent-3-en-1-ol

### Major Isomer (Ratio 4.4/1)

¹H-NMR (500.15 MHz, DMSO): 0.93 (d, 1H, J=6.8 Hz), 1.03 (t, 3H, J=7.1 Hz), 1.04 (t, 3H, J=7.1 Hz), 2.26 (s, 3H), 2.37-2.45 (m, 1H), 3.16-3.48 (m, 4H), 4.34 (dd, 1H, J=6.2 Hz, J=4.6 Hz), 4.69 (d, 1H, J=5.5 Hz), 5.15 (d, 1H, J=4.5 Hz), 5.20 (ddd, 1H, J=15.7 Hz, J=5.4 Hz, J=1.0 Hz), 5.65 (dd, 1H, J=15.8 Hz, J=7.7 Hz), 7.08 (d, 2H, J=7.9 Hz), 7.13 (d, 2H, J=8.0 Hz).

¹³C NMR (125 MHz, DMSO): δ 15.0, 15.1, 15.3, 20.6, 43.3, 59.8, 59.8, 75.9, 100.8, 126.5, 127.1, 128.1, 135.4, 136.5, 141.3.

### Minor Isomer

¹H-NMR (500.15 MHz, DMSO): 0.84 (d, 1H, J=6.9 Hz), 1.07 (t, 3H, J=7.1 Hz), 1.07 (t, 3H, J=7.1 Hz), 2.27 (s, 3H), 2.37-2.44 (m, 1H), 3.16-3.48 (m, 4H), 4.33-4.36 (m, 1H), 4.75 (d, 1H, J=5.4 Hz), 5.14 (d, 1H, J=4.4 Hz), 5.23 (ddd, 1H, J=15.7 Hz, J=5.5 Hz, J=1.0 Hz), 5.77 (dd, 1H, J=15.9 Hz, J=7.4 Hz), 7.09 (d, 2H, J=7.9 Hz), 7.14 (d, 2H, J=8.0 Hz).

¹³C NMR (125 MHz, DMSO): δ 15.1, 15.1, 16.4, 20.6, 43.1, 59.9, 59.9, 75.9, 101.0, 126.3, 127.2, 128.1, 136.1, 141.3.

If crotonaldehyde was used for the cross metathesis reaction with 2-methyl-1-(p-tolyl)but-3-en-1-ol in EtOAc (50° C.) only a low amount (2%) of desired product ((E)-2-methyl-5-oxo-1-(p-tolyl)pent-3-en-1-yl acetate) was observed by using 4 mol % GreenCat. Furthermore the formation 2% of (E)-2-methyl-1-(p-tolyl)pent-3-en-1-ol was observed. The usage of 3,3-diethoxyprop-1-ene with 2-methyl-1-(p-tolyl)but-3-en-1-ol in EtOAc (50° C.) gives with 4 mol % GreenCat 25% of the desired product (E)-5,5-diethoxy-2-methyl-1-(p-tolyl)pent-3-en-1-ol.

(E)-5,5-diethoxy-2-methyl-1-(p-tolyl)pent-3-en-1-ol can be transformed to (2E,4E)-4-methyl-5-(p-tolyl)penta-2,4-dienal in the presence of an acid (pTsOH, oxalic acid, tartaric acid, KHSO₄) under Dean-Stark conditions (toluene, or cyclohexane).

(E)-5,5-diethoxy-2-methyl-1-(p-tolyl)pent-3-en-1-ol can be deprotected in the presence of AcOH/water/THF (room temperature, 40 min) to (E)-5-hydroxy-4-methyl-5-(p-tolyl)pent-2-enal in high yield:

(E)-5,5-diethoxy-2-methyl-1-(p-tolyl)pent-3-en-1-ol (46 mg, 0.165 mmol) was stirred in 0.5 mL THF and 50 mg AcOH and 50 mg water were added. After 40 min at room temperature the solvent was evaporated under reduced pressure. 5 mL heptane were added (twice) and were evaporated under reduced pressure. 32 mg (0.157 mmol, 95% yield) of pure (E)-5-hydroxy-4-methyl-5-(p-tolyl)pent-2-enal were obtained.

### (E)-5-hydroxy-4-methyl-5-(p-tolyl)pent-2-enal

### Major Isomer (Ratio 4.4/1)

¹H-NMR (500.15 MHz, DMSO): 0.96 (d, 1H, J=6.8 Hz), 2.27 (s, 3H), 2.73-2.79 (m, 1H), 4.59 (dd, 1H, J=4.9 Hz, J=4.9 Hz), 5.41 (d, 1H, J=4.5 Hz), 5.97 (ddd, 1H, J=15.7 Hz, J=7.9 Hz, J=1.2 Hz), 7.00 (dd, 1H, J=15.8 Hz, J=7.1 Hz), 7.14-7.24 (m, 4H), 9.44 (d, J=8.0 Hz).

¹³C NMR (125 MHz, DMSO): 513.7, 20.6, 43.8, 74.6, 126.3, 128.2, 131.8, 135.7, 140.4, 161.9, 194.5.

### Minor Isomer

¹H-NMR (500.15 MHz, DMSO): 0.92 (d, 1H, J=6.9 Hz), 2.28 (s, 3H), 2.68-2.75 (m, 1H), 4.46-4.51 (m, 1H), 5.42 (d, 1H, J=4.5 Hz), 5.99 (ddd, 1H, J=15.7 Hz, J=7.8 Hz, J=1.2 Hz), 7.07 (dd, 1H, J=15.7 Hz, J=7.5 Hz), 7.14-7.24 (m, 4H), 9.47 (d, J=8.0 Hz).

¹³C NMR (125 MHz, DMSO): 515.8, 20.6, 44.1, 75.4, 126.3, 128.4, 132.1, 135.9, 140.8, 161.9, 194.5.

(E)-5-hydroxy-4-methyl-5-(p-tolyl)pent-2-enal can be transformed to (2E,4E)-4-methyl-5-(p-tolyl)penta-2,4-dienal in the presence of an acid (pTsOH, oxalic acid, tartaric acid, KHSO₄) under Dean-Stark conditions (toluene, or cyclohexane).

(E)-5-hydroxy-4-methyl-5-(p-tolyl)pent-2-enal can be transformed to ((2E,4E)-4-methyl-5-(p-tolyl)penta-2,4-dienal in the presence of POCl₃/Pyridine (0° C. to RT).

Smaller amounts of ((2E,4Z)-4-methyl-5-(p-tolyl)penta-2,4-dienal were also formed.

¹³C NMR (125 MHz, CDCl₃): δ 20.5, 21.3, 129.2, 129.5, 130.0, 132.6, 133.4, 138.1, 139.1, 150.6, 194.4.

### Example 16

### Cross Metathesis Between 2-methyl-1-(p-tolyl)but-3-en-1-yl acetate and 3,3-diethoxyprop-1-ene to Prepare (E)-5,5-diethoxy-2-methyl-1-(p-tolyl)pent-3-en-1-yl acetate

To a stirred solution of 2-methyl-1-(p-tolyl)but-3-en-1-yl acetate (1.0 g, 4.581 mmol) and 3,3-diethoxyprop-1-ene (1.79 g, 13.74 mmol) in EtOAc (8 ml) at 50° C. was added portion wise (5 times 0.3 mol % each hour) 1.5 mol % GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6) over 5 hours. After the last addition the mixture was cooled to room temperature and stirred overnight. Then 100 mg SnatchCat® (1,4-Bis(2-isocyanopropyl)piperazine, CAS 51641-96-4) were added and the mixture was stirred for 30 minutes at room temperature. The mixture was filtered through a pad of silica and the solvent was evaporated under reduced pressure (50 mbar, 50° C.). The crude was purified by column chromatography (40 g cartridge, from Cyclohexane 98/MTBE 2 to Cyclohexane 9/AcOEt 1) and 0.73 g (2.28 mmol, 50% yield) of (E)-5,5-diethoxy-2-methyl-1-(p-tolyl)pent-3-en-1-yl acetate were obtained. Unreacted 3,3-diethoxyprop-1-ene, 2-methyl-1-(p-tolyl)but-3-en-1-yl acetate (0.280 g, 1.28 mmol, 28% recycling yield) and (E)-1,1,4,4-tetraethoxybut-2-ene could be also isolated. The formation of (E)-2,5-dimethyl-1,6-di-p-tolylhex-3-ene-1,6-diyl diacetate was only observed in traces.

### (E)-5,5-diethoxy-2-methyl-1-(p-tolyl)pent-3-en-1-yl acetate

### Major Isomer (Ratio 1.46/1)

¹H-NMR (500.15 MHz, DMSO): 0.97 (d, 1H, J=6.9 Hz), 1.01 (t, 3H, J=7.1 Hz), 1.04 (t, 3H, J=7.1 Hz), 2.03 (s, 3H), 2.27 (s, 3H), 2.63-2.74 (m, 1H), 3.13-3.56 (m, 4H), 4.70 (d, 1H, J=5.1 Hz), 5.28 (ddd, 1H, J=15.7 Hz, J=5.4 Hz, J=1.0 Hz), 5.52 (d, 1H, J=7.0 Hz), 5.58 (ddd, 1H, J=15.8 Hz, J=7.7 Hz, J=1.0 Hz), 7.10-7.20 (m, 4H).

¹³C NMR (125 MHz, DMSO): δ 15.0, 15.0, 15.5, 20.6, 20.7, 40.8, 59.8, 59.9, 77.9, 100.5, 126.8, 128.5, 128.7, 134.3, 136.0, 136.7, 169.5.

### Minor Isomer

¹H-NMR (500.15 MHz, DMSO): 0.83 (d, 1H, J=6.9 Hz), 1.09 (t, 6H, J=7.1 Hz), 2.00 (s, 3H), 2.28 (s, 3H), 2.63-2.74 (m, 1H), 3.13-3.56 (m, 4H), 4.78 (d, 1H, J=5.1 Hz), 5.39 (ddd, 1H, J=15.7 Hz, J=5.4 Hz, J=1.0 Hz), 5.51 (d, 1H, J=7.0 Hz), 5.67 (ddd, 1H, J=15.8 Hz, J=7.7 Hz, J=1.0 Hz), 7.10-7.20 (m, 4H).

¹³C NMR (125 MHz, DMSO): δ 15.0, 15.1, 16.4, 20.6, 20.7, 41.2, 60.4, 60.4, 78.0, 100.7, 126.7, 128.7, 128.7, 134.6, 136.0, 136.8, 169.4.

If crotonaldehyde was used for the cross metathesis reaction with 2-methyl-1-(p-tolyl)but-3-en-1-yl acetate in EtOAc (50° C.) only a low amount (23.8%) of desired product ((E)-2-methyl-5-oxo-1-(p-tolyl)pent-3-en-1-yl acetate) was observed by using 2 mol % GreenCat. Furthermore the formation of 21% of (E)-2-methyl-1-(p-tolyl)pent-3-en-1-yl acetate was observed.

(E)-5,5-diethoxy-2-methyl-1-(p-tolyl)pent-3-en-1-yl acetate can be transformed to (2E,4E)-4-methyl-5-(p-tolyl)penta-2,4-dienal in the presence of an acid or a Lewis acid (5 mol % pTsOH at 50° C. or 10 mol % BF₃·Et₂O at room temperature, in toluene or cyclohexane). 1-((1E,3E)-5,5-diethoxy-2-methylpenta-1,3-dien-1-yl)-4-methylbenzene is observed as an intermediate of the reaction.

Smaller amounts of ((2E,4Z)-4-methyl-5-(p-tolyl)penta-2,4-dienal were also formed.

¹³C NMR (125 MHz, CDCl₃): δ 20.5, 21.3, 129.2, 129.5, 130.0, 132.6, 133.4, 138.1, 139.1, 150.6, 194.4.

(E)-5,5-diethoxy-2-methyl-1-(p-tolyl)pent-3-en-1-yl acetate can be deprotected in the presence of AcOH/water/THF (room temperature, 30 min) to (E)-2-methyl-5-oxo-1-(p-tolyl)pent-3-en-1-yl acetate in quantitative yield.

### (E)-2-methyl-5-oxo-1-(p-tolyl)pent-3-en-1-yl acetate

### Major Isomer (Ratio 1.37/1)

¹H-NMR (500.15 MHz, DMSO): 1.04 (d, 1H, J=6.9 Hz), 2.07 (s, 3H), 2.27 (s, 3H), 2.96-3.08 (m, 1H), 5.73 (d, 1H, J=6.5 Hz), 6.00 (ddd, 1H, J=15.7 Hz, J=7.8 Hz, J=1.2 Hz), 6.92 (dd, 1H, J=15.8 Hz, J=7.3 Hz), 7.14-7.24 (m, 4H), 9.43 (d, J=7.8 Hz).

¹³C NMR (125 MHz, DMSO): 914.3, 20.6, 20.7, 41.3, 76.7, 126.5, 128.7, 132.7, 135.3, 137.0, 159.1, 169.5, 194.4.

### Minor Isomer

¹H-NMR (500.15 MHz, DMSO): 0.93 (d, 1H, J=6.9 Hz), 2.02 (s, 3H), 2.28 (s, 3H), 2.96-3.08 (m, 1H), 5.64 (d, 1H, J=7.4 Hz), 6.08 (ddd, 1H, J=15.7 Hz, J=7.8 Hz, J=1.2 Hz), 6.98 (dd, 1H, J=15.8 Hz, J=7.8 Hz), 7.14-7.24 (m, 4H), 9.49 (d, J=7.9 Hz).

¹³C NMR (125 MHz, DMSO): 515.5, 20.6, 20.7, 41.6, 77.4, 126.6, 128.8, 132.8, 135.6, 137.2, 159.3, 169.4, 194.4.

(E)-2-methyl-5-oxo-1-(p-tolyl)pent-3-en-1-yl acetate can be transformed to (2E,4E)-4-methyl-5-(p-tolyl)penta-2,4-dienal in the presence of an acid (pTsOH, oxalic acid, tartaric acid, KHSO₄) under Dean-Stark conditions (toluene, or cyclohexane).

(E)-2-methyl-5-oxo-1-(p-tolyl)pent-3-en-1-yl acetate can be deprotected to (E)-5-hydroxy-4-methyl-5-(p-tolyl)pent-2-enal in the presence of KOH/MeOH (quantitative yield).

(E)-5-hydroxy-4-methyl-5-(p-tolyl)pent-2-enal can be transformed to (2E,4E)-4-methyl-5-(p-tolyl)penta-2,4-dienal in the presence of an acid (pTsOH, oxalic acid, tartaric acid, KHSO₄) under Dean-Stark conditions (toluene, or cyclohexane).

(E)-5-hydroxy-4-methyl-5-(p-tolyl)pent-2-enal can be transformed to ((2E,4E)-4-methyl-5-(p-tolyl)penta-2,4-dienal in the presence of POCl₃/Pyridine (0° C. to RT).

Smaller amounts of ((2E,4Z)-4-methyl-5-(p-tolyl)penta-2,4-dienal were also formed.

¹³C NMR (125 MHz, CDCl₃): δ 20.5, 21.3, 129.2, 129.5, 130.0, 132.6, 133.4, 138.1, 139.1, 150.6, 194.4.

### Example 17

### Preparation of 2-methyl-1-(p-tolyl)but-3-en-2-ol from 4-methylphenylacetone

To a cooled solution (0° C.) of 139.9 mL vinylmagnesium chloride (1.6 M in THF, 254.4 mmol, 1.1 eq) was added slowly a solution of 1-(p-tolyl)propan-2-one (37.7 g, 254.4 mmol) in 153 mL THF. The internal temperature did not exceed 5° C. during the addition of the ketone. The mixture was further stirred at 0° C. over night (16 hours). The reaction mixture was added slowly to a cooled solution of 18.3 g AcOH (305.2 mmol) in 200 ml water. The phases were separated and the aqueous phase was extracted with 150 mL TBME. The combined organic phase were washed with a saturated aqueous NaHCO₃ solution and a saturated aqueous NaCl solution. After drying over Na₂SO₄ the solvent was evaporated under reduced pressure (500-20 mbar, 50° C.). The crude (48.2 g) was purified by a distillation through a Vigreux column under reduced pressure (oilbath 40° C.-125° C., 50-4 mbar, bp 97° C./4 mbar). 35.5 g (201.4 mmol, 79% yield) 2-methyl-1-(p-tolyl)but-3-en-2-ol of a colourless liquid were obtained.

### 2-methyl-1-(p-tolyl)but-3-en-2-ol

¹H-NMR analysis results in CDCl₃ were in accordance with data from literature:
Araki, S.; Ohmura, M.; Butsugan, Y. Bulletin of the Chemical Society of Japan (1986), 59(6), 2019-20.

¹³C NMR (100 MHz, CDCl₃): 521.0, 27.4, 48.3, 73.0, 111.9, 128.8, 130.5, 133.7, 136.1, 144.8.

### Example 18

### Cross Metathesis Between 2-methyl-1-(p-tolyl)but-3-en-2-ol and 3,3-diethoxyprop-1-ene to Prepare (E)-5,5-diethoxy-2-methyl-1-(p-tolyl)pent-3-en-2-ol

To a stirred solution of 2-methyl-1-(p-tolyl)but-3-en-2-ol (1.6 g, 9.078 mmol) and 3,3-diethoxyprop-1-ene (3.77 g, 27.2 mmol) in EtOAc (16 ml) at 50° C. was added portion wise (0.2 mol % each hour during 5 hours) GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6). Then 0.5 mol % GreenCat were added and the mixture was stirred over night at room temperature. The mixture was heated again to 50° C. and two times 0.5 mol % GreenCat were added after 2 hours of heating (overall reaction time 30 hours, overall GreenCat added: 178 mg, 2.5 mol %). The heating was stopped, then 187 mg SnatchCat® (1,4-Bis(2-isocyanopropyl)piperazine, CAS 51641-96-4) were added and the mixture was stirred for 30 minutes at room temperature. The mixture was filtered through a pad of silica and the solvent was evaporated under reduced pressure (50 mbar, 50° C.). The crude was purified by column chromatography (40 g cartridge, from Cyclohexane 95/MTBE 5 to Cyclohexane 9/AcOEt 1) and 1.57 g (5.64 mmol, 62% yield) of (E)-5,5-diethoxy-2-methyl-1-(p-tolyl)pent-3-en-2-ol were obtained. Unreacted 3,3-diethoxyprop-1-ene, 2-methyl-1-(p-tolyl)but-3-en-2-ol (0.42 g, 2.389 mmol, 26% recycling yield) and (E)-1,1,4,4-tetraethoxybut-2-ene could be also isolated. The formation of (E)-2,5-dimethyl-1,6-di-p-tolylhex-3-ene-2,5-diol was not observed.

(E)-5,5-diethoxy-2-methyl-1-(p-tolyl)pent-3-en-2-ol ¹H-NMR (500.15 MHz, DMSO): 1.08 (t, 3H, J=7.1 Hz), 1.08 (t, 3H, J=7.1 Hz) 1.09 (s, 3H), 2.24 (s, 3H), 2.65 (s, 2H), 3.28-3.38 (m, 2H), 3.40-3.49 (m, 2H), 4.64 (br s OH, 1H), 4.80 (dd, 1H, J=5.5 Hz, J=0.8 Hz), 5.41 (dd, 1H, J=15.7 Hz, J=5.5 Hz), 5.80 (dd, 1H, J=15.8 Hz, J=0.9 Hz)), 7.01-7.06 (m, 4H).

¹³C NMR (125 MHz, DMSO): δ 15.1, 15.1, 20.6, 27.1, 47.9, 59.9, 60.0, 71.3, 100.8, 124.2, 127.9, 130.3, 134.5, 134.7, 141.1.

If crotonaldehyde was used for the cross metathesis reaction with 2-methyl-1-(p-tolyl)but-3-en-2-ol in EtOAc (50° C.) only a low amount (34%) of desired product ((E)-4-hydroxy-4-methyl-5-(p-tolyl)pent-2-enal) was observed by using 3 mol % GreenCat. Furthermore the formation of 21% of (E)-2-methyl-1-(p-tolyl)pent-3-en-2-ol was observed.

(E)-5,5-diethoxy-2-methyl-1-(p-tolyl)pent-3-en-2-ol can be transformed to (2E,4E)-4-methyl-5-(p-tolyl)penta-2,4-dienal in the presence of an acid (pTsOH, oxalic acid, tartaric acid, KHSO₄) under Dean-Stark conditions (toluene, or cyclohexane).

(E)-5,5-diethoxy-2-methyl-1-(p-tolyl)pent-3-en-2-ol can be deprotected in the presence of AcOH/water/heptane (room temperature, 30 min) to (E)-4-hydroxy-4-methyl-5-(p-tolyl)pent-2-enal in high yield:

(E)-5,5-diethoxy-2-methyl-1-(p-tolyl)pent-3-en-2-ol (200 mg, 0.718 mmol) was stirred in 1 mL heptane and 215 mg AcOH and 207 mg water were added. After 40 min at room temperature the solvent was evaporated under reduced pressure. 5 mL heptane were added and were evaporated under reduced pressure (the evaporation of 5 mL heptane was repeated a second time). 146 mg (0.715 mmol, 99% yield) of pure (E)-5-hydroxy-4-methyl-5-(p-tolyl)pent-2-enal were obtained.

### (E)-4-hydroxy-4-methyl-5-(p-tolyl)pent-2-enal

¹H-NMR (500.15 MHz, CDCl₃): 1.20 (s, 3H), 2.25 (s, 3H), 2.65 (s, 2H), 2.78 (d. J=2.1 Hz), 3.34 (s, 1H), 5.10 (br s OH, 1H), 6.03 (dd, 1H, J=15.6 Hz, J=8.0 Hz), 7.05 (d, 1H, J=15.5 Hz), 7.05 (d, 2H, J=8.0 Hz), 7.09 (d, 2H, J=8.1 Hz), 9.51 (d, J=8.1 Hz).

¹³C NMR (125 MHz, CDCl₃): 521.0, 27.1, 47.7, 73.1, 129.2, 129.8, 130.3, 132.2, 136.9, 162.7, 193.6.

(E)-4-hydroxy-4-methyl-5-(p-tolyl)pent-2-enal can be transformed to (2E,4E)-4-methyl-5-(p-tolyl)penta-2,4-dienal in the presence of an acid (pTsOH, oxalic acid, tartaric acid, KHSO₄) under Dean-Stark conditions (toluene, or cyclohexane).

(E)-4-hydroxy-4-methyl-5-(p-tolyl)pent-2-enal can be transformed to ((2E,4E)-4-methyl-5-(p-tolyl)penta-2,4-dienal in the presence of POCl₃/Pyridine (0° C. to RT).

Smaller amounts of ((2E,4Z)-4-methyl-5-(p-tolyl)penta-2,4-dienal were also formed.

### Example 19

### Cross Metathesis Between 2-methyl-1-(p-tolyl)but-3-en-1-yl acetate and prop-2-ene-1,1-diyl diacetate to Prepare (E)-4-methyl-5-(p-tolyl)pent-2-ene-1,1,5-triyl triacetate

To a stirred solution of 2-methyl-1-(p-tolyl)but-3-en-1-yl acetate (1.0 g, 4.581 mmol) and prop-2-ene-1,1-diyl diacetate (2.2 g, 13.74 mmol) in EtOAc (8 ml) at 50° C. was added portion wise (1 mol % each hour) 2 mol % GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6) over 2 hours. After the last addition the mixture was cooled to room temperature and stirred overnight. 2 mol % GreenCat were added and the mixture was stirred 90 hours at room temperature. Then 472 mg SnatchCat® (1,4-Bis(2-isocyanopropyl)piperazine, CAS 51641-96-4) were added and the mixture was stirred for 30 minutes at room temperature. The solvent was evaporated under reduced pressure (50 mbar, 50° C.). The crude (4.21 g) was purified by column chromatography (40 g cartridge, from Cyclohexane 95/MTBE 5 to Cyclohexane 6/MTBE 4) and 0.230 g (0.66 mmol, 14.4% yield) of (E)-4-methyl-5-(p-tolyl)pent-2-ene-1,1,5-triyl triacetate and (E)-but-2-ene-1,1,4,4-tetrayl tetraacetate (130 mg) were obtained. Unreacted prop-2-ene-1,1-diyl diacetate (1.68 g), 2-methyl-1-(p-tolyl)but-3-en-1-yl acetate (0.722 g, 3.31 mmol, 72% recycling yield) and (E)-1,1,4,4-tetraethoxybut-2-ene could be also isolated. The formation of (E)-2,5-dimethyl-1,6-di-p-tolylhex-3-ene-1,6-diyl diacetate and was only observed in traces.

### (E)-4-methyl-5-(p-tolyl)pent-2-ene-1,1,5-triyl triacetate

¹³C NMR (125 MHz, DMSO): δ 14.5, 20.4, 20.4, 20.6, 20.7, 40.5, 77.3, 88.6, 124.2, 126.5, 128.5, 135.6, 136.7, 137.8, 168.2, 168.3, 169.5.

### Minor Isomer (Characteristic Signals):

¹³C NMR (125 MHz, DMSO): δ 15.7, 20.4, 20.6, 41.0, 77.7, 88.7, 124.4, 126.8, 128.7, 135.7, 137.0, 138.1, 168.2, 168.3, 169.3.

(E)-4-methyl-5-(p-tolyl)pent-2-ene-1,1,5-triyl triacetate can be deprotected to (E)-5-hydroxy-4-methyl-5-(p-tolyl)pent-2-ene-1,1-diyl diacetate in the presence of KOH, MeOH and water at room temperature. (E)-5-hydroxy-4-methyl-5-(p-tolyl)pent-2-ene-1,1-diyl diacetate can be deprotected to (E)-5-hydroxy-4-methyl-5-(p-tolyl)pent-2-enal in the presence of 1 eq triethylamine in MeOH (4 hours at room temperature).

### Example 20

### Cross Metathesis Between 1-(tert-butyl)-4-vinylbenzene and 3,3-diethoxyprop-1-ene to Prepare (E)-1-(tert-butyl)-4-(3,3-diethoxyprop-1-en-1-yl)benzene

To a stirred solution of 0.50 g 1-(tert-butyl)-4-vinylbenzene (94% purity, 2.933 mmol) and 1.22 g 3,3-diethoxyprop-1-ene (8.798 mmol, 3 eq) in 10 mL EtOAc at 50° C. under Argon atmosphere was added 10 mg (0.0127 mmol, 0.43 mol %) GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6). The mixture was stirred for 30 minutes at 50° C. Then twice 10 mg (0.0127 mmol, 0.43 mol %) GreenCat were added every 30 minutes and the mixture was stirred further 30 min. After the addition of 70 mg SnatchCat® (1,4-Bis(2-isocyanopropyl)piperazine, CAS 51641-96-4) and 30 minutes of stirring the solvent was evaporated under reduced pressure. The crude was purified by column chromatography (80 g cartridge, from Cyclo 95/AcOEt 5 to Cyclo 9/AcOEt 1) and 371 mg (1.341 mmol, 45.7% yield) (E)-1-(tert-butyl)-4-(3,3-diethoxyprop-1-en-1-yl)benzene, 110 mg (0.584 mmol, 19.9% yield, the deprotection was observed during the column chromatography) (E)-3-(4-(tert-butyl)phenyl)acrylaldehyde and 0.45 g (1.84 mmol) (E)-1,1,4,4-tetraethoxybut-2-ene were obtained. Unreacted 3,3-diethoxyprop-1-ene and 1-(tert-butyl)-4-vinylbenzene (26.3 mg, 0.164 mmol, 5.6% recycled yield) could be also isolated. Formation of 97 mg (0.332 mmol, 22% recycling yield) (E)-1,2-bis(4-(tert-butyl)phenyl)ethene was also observed.

### (E)-1-(tert-butyl)-4-(3,3-diethoxyprop-1-en-1-yl)benzene

¹H-NMR (500.15 MHz, CDCl₃): 1.24 (t, 6H, J=7.0 Hz), 1.31 (s, 9H), 3.51-3.58 (m, 2H), 3.66-3.73 (m, 2H), 5.06 (dd, 1H, J=5.4 Hz, J=1.0 Hz), 6.16 (dd, 1H, J=16.1 Hz, J=5.2 Hz), 6.68 (d, 1H, J=16.0 Hz), 7.34 (s, 4H).

¹³C NMR (100 MHz, CDCl₃): 915.5, 31.3, 34.6, 61.0, 101.63, 125.5, 125.9, 126.5, 132.7, 133.4, 151.2.

### (E)-3-(4-(tert-butyl)phenyl)acrylaldehyde

¹³C NMR (100 MHz, CDCl₃): δ 31.1, 35.0, 126.1, 128.0, 128.4, 131.3, 152.8, 155.1, 193.8.

### (E)-1,2-bis(4-(tert-butyl)phenyl)ethene

¹³C NMR (100 MHz, CDCl₃): 531.3, 34.6, 125.6, 126.1, 127.7, 134.8, 150.5.

(E)-1-(tert-butyl)-4-(3,3-diethoxyprop-1-en-1-yl)benzene can be transformed to (E)-3-(4-(tert-butyl)phenyl)acrylaldehyde in the presence of water and AcOH (80° C.).

### Example 21

### Cross Metathesis Between 1-(tert-butyl)-4-vinylbenzene and prop-2-ene-1,1-diyl diacetate to Prepare (E)-3-(4-(tert-butyl)phenyl)prop-2-ene-1,1-diyl diacetate

To a stirred solution of 0.60 g 1-(tert-butyl)-4-vinylbenzene (94% purity, 3.519 mmol mmol) and 2.369 g prop-2-ene-1,1-diyl diacetate (14.976 mmol, 4 eq) in 15 mL dichloromethane at 40° C. (reflux) under Argon atmosphere was added 37 mg (0.047 mmol, 1.3 mol %) GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6). The mixture was stirred for 24 hours (reflux). Then 37 mg (0.047 mmol, 1.3 mol %) GreenCat were added and the mixture was stirred for another 24 hours (reflux). After the addition of 103 mg (0.468 mmol) SnatchCat® (1,4-Bis(2-isocyanopropyl)piperazine, CAS 51641-96-4) and 30 minutes of stirring at room temperature the solvent was evaporated under reduced pressure. The crude was purified by column chromatography (80 g cartridge, from Cyclo 95/AcOEt 5 to Cyclo 9/AcOEt 1) and 423 mg (1.46 mmol, 41.4% yield) of (E)-3-(4-(tert-butyl)phenyl)prop-2-ene-1,1-diyl diacetate were obtained. Unreacted prop-2-ene-1,1-diyl diacetate (1.55 g) and 1-(tert-butyl)-4-vinylbenzene (112 mg, 0.698 mmol, 20% recycled yield) could be also isolated. The formation of 0.148 g (0.506 mmol, 29% recycled yield) (E)-1,2-bis(4-(tert-butyl)phenyl)ethene was also observed.

### (E)-3-(4-(tert-butyl)phenyl)prop-2-ene-1,1-diyl diacetate

¹H-NMR (500.15 MHz, CDCl₃): 1.31 (s, 9H), 2.11 (s, 6H), 6.16 (dd, 1H, J=16.0 Hz, J=6.5 Hz), 6.68 (d, 1H, J=16.0 Hz), 7.29 (d, 1H, J=6.5 Hz). 7.35 (s, 4H).

¹³C NMR (150 MHz, CDCl₃): 520.9, 31.2, 34.7, 89.9, 120.9, 125.6, 126.8, 132.4, 135.5, 152.1, 168.7.

Deprotection was done according to J. Indian Chem. Soc., Vol. 76, November-December 1999 (Titanium isopropoxide).

### Example 22

### Cross Metathesis Between 2-methyl-1-(4-vinylphenyl)propan-2-ol and 3,3-diethoxyprop-1-ene to Prepare (E)-1-(4-(3,3-diethoxyprop-1-en-1-yl)phenyl)-2-methylpropan-2-ol

To a stirred solution of 2-methyl-1-(4-vinylphenyl)propan-2-ol (1.0 g, 5.67 mmol) and 3,3-diethoxyprop-1-ene (2.2 g, 17.02 mmol) in EtOAc (10 ml) at 50° C. was added portion wise (3 times 1 mol % each hour) 133.6 mg (3 mol %) GreenCat ((1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), Apeiron CAS 1448663-06-6) over 3 hours. After the last addition the mixture was cooled to room temperature and stirred overnight. Then 246 mg SnatchCat® (1,4-Bis(2-isocyanopropyl)piperazine, CAS 51641-96-4) were added and the mixture was stirred for 30 minutes at room temperature. The mixture was filtered through a pad of silica and the solvent was evaporated under reduced pressure (50 mbar, 50° C.). The crude (3.05 g) was purified by column chromatography (40 g cartridge, from Cyclohexane 98/MTBE 2 to Cyclohexane 9/AcOEt 1) and 0.86 g (3.1 mmol, 54% yield) of (E)-1-(4-(3,3-diethoxyprop-1-en-1-yl)phenyl)-2-methylpropan-2-ol were obtained. Unreacted 3,3-diethoxyprop-1-ene (0.5 g), 2-methyl-1-(4-vinylphenyl)propan-2-ol (0.140 g, 0.79 mmol, 14% recycling yield) and (E)-1,1,4,4-tetraethoxybut-2-ene (630 mg) could be also isolated. Also 180 mg (0.77 mmol, 27% recycling yield) (E)-1,1′-(ethene-1,2-diylbis(4,1-phenylene))bis(2-methylpropan-2-ol) were isolated.

### (E)-1-(4-(3,3-diethoxyprop-1-en-1-yl)phenyl)-2-methylpropan-2-ol

¹H-NMR (500.15 MHz, DMSO): 1.05 (s, 6H), 1.14 (t, 6H, J=7.0 Hz), 2.63 (s, 2H), 3.45-3.51 (m, 2H), 3.57-3.63 (m, 2H), 4.31 (br s OH, 1H), 4.03 (dd, 1H, J=5.4 Hz, J=0.5 Hz), 6.19 (dd, 1H, J=16.1 Hz, J=5.4 Hz), 6.63 (d, 1H, J=16.1 Hz), 7.17 (d, 2H, J=8.0 Hz), 7.37 (d, 2H, J=8.1 Hz).

¹³C NMR (150 MHz, CDCl₃): 515.3, 29.2, 49.5, 61.1, 70.8, 101.6, 126.3, 126.6, 130.7, 132.6, 134.5, 137.8.

### (E)-1,1′-(ethene-1,2-diylbis(4,1-phenylene))bis(2-methylpropan-2-ol)

¹³C NMR (125 MHz, DMSO): 929.1, 49.1, 69.3, 125.5, 127.4, 130.6, 134.7, 138.3.

(E)-1-(4-(3,3-diethoxyprop-1-en-1-yl)phenyl)-2-methylpropan-2-ol can be deprotected in the presence of AcOH/water (room temperature, 30 min) to (E)-3-(4-(2-hydroxy-2-methylpropyl)phenyl)acrylaldehyde in quantitative yield.

### (E)-3-(4-(2-hydroxy-2-methylpropyl)phenyl)acrylaldehyde

¹³C NMR (125 MHz, CDCl₃): 529.3, 49.6, 70.9, 128.1, 128.3, 131.3, 132.2, 142.0, 152.9, 193.9.

1. A process for the preparation of a compound of formula (I) 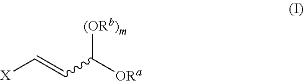
in the form of any one of its stereoisomers or a mixture thereof, and wherein m is 0 or 1; R^{a} is a C₁₋₄ alkyl group, a trimethyl silyl group, a C(═O)—R^{c} group, a C(═O)—OR^{c} group, a CH₂(OR^{c}) group, a CH(OR^{c})CH₃ group wherein R^{c} is a C₁₋₄ alkyl group; R^{b} is a C₁₋₄ alkyl group, a C(═O)—R^{c} group wherein R^{c} is a C₁₋₄ alkyl group; or R^{a} and R^{b} are taken together and represent a C₂₋₆ alkanediyl group and X is a group of formula a), b) or c); 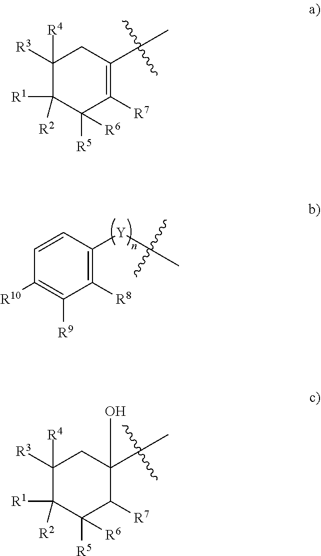
in the form of any one of its stereoisomers, and wherein each R¹, R², R³, R⁴, R⁵, R⁶ and R⁷, independently from each other, represent a hydrogen atom, a C₁₋₆ alkyl group or a C₂₋₆ alkenyl group, each optionally substituted by a hydroxy or C₁₋₃ alkoxy group; or two groups among R¹, R², R³, R⁴, R⁵, R⁶ and R⁷ are taken together and form C₃₋₈ cycloalkyl or C₅₋₈ cycloalkenyl group, each optionally substituted by a hydroxy, C₁₋₃ alkyl or C₁₋₃ alkoxy group, and the others groups have the same meaning as defined above; each R⁸, R⁹ and R¹⁰, independently from each other, represent a hydrogen atom, a C₁₋₆ alkyl group or a C₂₋₆ alkenyl group, each optionally substituted by a hydroxy or C₁₋₃ alkoxy group; or two groups among R⁸, R⁹ and R¹⁰ are taken together and form C₃₋₈ cycloalkyl or C₅₋₈ cycloalkenyl group, each optionally substituted by a hydroxy, C₁₋₃ alkyl or C₁₋₃ alkoxy group, and the other group has the same meaning as defined above; n is 0 or 1 and Y is a —CH═CR¹¹—, —CHR¹²—CHR¹¹— or —CH₂—C(OH)R¹¹— group wherein R¹¹ is a hydrogen atom or a methyl or ethyl group and R¹² is a hydroxy or a acetate group;
provided that m is 1 when X is a group of formula b);
comprising a cross metathesis step, in the presence of a metathesis catalyst, between compound of the formula (IIa), (IIb) or (IIc): 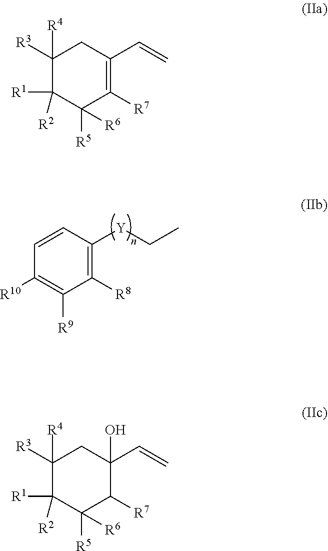
in the form of any one of its stereoisomers, and wherein each R, R², R³, R⁴, R⁵, R⁶, R⁷, R⁸, R⁹, R¹⁰, n and Y have the same meaning as defined above
with compound of formula (III); 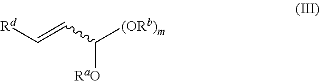
in the form of any one of its stereoisomers or a mixture thereof, and wherein m is 0 or 1 when the cross metathesis step is performed with compound of the formula (IIa) or (IIc) or m is 1 when the cross metathesis step is performed with compound of the formula (IIb), R^{a} is a C₁₋₄ alkyl group, a trimethyl silyl group, a C(═O)—R^{c} group, a C(═O)—OR^{c} group, a CH₂(OR^{c}) group, a CH(OR^{c})CH₃ group wherein R^{c} is a C₁₋₄ alkyl group; R^{b} is a C₁₋₄ alkyl group, a C(═O)—R^{c} group wherein R^{c} is a C₁₋₄ alkyl group; or R^{a} and R^{b} are taken together and represent a C₂₋₆ alkanediyl group, R^{d} is a hydrogen atom or a CH(OR^{a})(OR^{b})ₘ group wherein m, R^{a} and R^{b} have the same meaning as defined above.

2. The process according to claim 1, wherein R³, R⁴, R⁵, R⁶, R⁷, R⁸ and R⁹ independently from each other, represent a hydrogen atom or a C₁₋₃ alkyl group.

3. The process according to claim 1, wherein X is a group of formula a) or c).

4. The process according to claim 1, wherein the compound of formula (I) is of formula (I″): 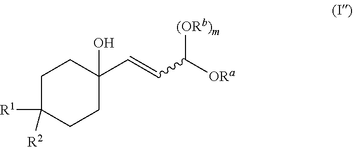
in the form of any one of its stereoisomers or a mixture thereof, and wherein m, R^{a}, R^{b}, R¹ and R² have the same meaning as defined in claim 1;
and said compound of formula (IIc) is of formula (IIc′) 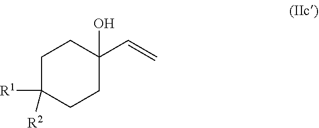
in the form of any one of its stereoisomers or a mixture thereof, and wherein each R¹ and R² have the same meaning as defined in claim 1.

5. The process according to claim 1, wherein R¹ is a C₁₋₄ alkyl group or a C₂₋₄ alkenyl group.

6. The process according to claim 1, wherein R¹ is a methyl group.

7. The process according to claim 11, wherein R² is a hydrogen atom, a C₁₋₃ alkyl group or a C₂₋₃ alkenyl group.

8. The process according to claim 1, wherein R² is a methyl group.

9. The process according to claim 1, wherein R^{d} is a hydrogen atom and m is 1.

10. The process according to claim 1, wherein the metathesis catalyst is a Ruthenium-based catalyst.

11. The process according to claim 1, wherein the metathesis catalyst is selected from the group consisting of 1,3-bis(2,6-diisopropylphenyl)imidazolidin-2-ylidene)dichloro(2-((1-(methoxy(methyl)amino)-1-oxopropan-2-yl)oxy)benzylidene)ruthenium(II), (1,3-bis(2,6-diisopropylphenyl)imidazolidin-2-ylidene)diiodo(2-((1-(methoxy(methyl)amino)-1-oxopropan-2-yl)oxy)benzylidene)ruthenium(II), (1,3-dimesitylimidazolidin-2-ylidene)dichloro(2-isopropoxy-5-nitrobenzylidene)ruthenium(II), Dichloro[1,3-bis(2,4,6-trimethylphenyl)-2-imidazolidinylidene](2-isopropoxyphenylmethylene)ruthenium(II), Benzylidene-bis(tricyclohexylphosphine)dichlororuthenium, Dichloro[1,3-bis(2,6-isopropylphenyl)-2-imidazolidinylidene](2-isopropoxyphenylmethylene)ruthenium(II), [1,3-Bis(2,6-di-i-propylphenyl)imidazolidin-2-ylidene)(2-i-propoxy-5-nitrobenzylidene) ruthenium(II) diiodide, 1,3-Bis(2,6-di-i-propylphenyl)imidazolidin-2-ylidene)(2-i-propoxy-5-nitrobenzylidene) ruthenium(II) dichloride, (1,3-Dimesitylimidazolidin-2-ylidene)diiodo(2-isopropoxy-5-nitrobenzylidene)ruthenium(II), Bis(1-(2,6-diethylphenyl)-3,5,5-trimethyl-3-phenylpyrrolidin-2-ylidene)(3-phenyl-1H-inden-1-ylidene)ruthenium(II) dichloride, (1-(2,6-diethylphenyl)-3,5,5-trimethyl-3-phenylpyrrolidin-2-ylidene)dichloro(2-isopropoxy-5-nitrobenzylidene)ruthenium(II), (1-(2,6-diethylphenyl)-3,5,5-trimethyl-3-phenylpyrrolidin-2-ylidene)diiodo(2-isopropoxy-5-nitrobenzylidene)ruthenium(II), Dichloro[1,3-bis(2-methylphenyl)-2-imidazolidinylidene](2-isopropoxyphenylmethylene)ruthenium(II), 1,3-Bis(2,4,6-trimethylphenyl)-4,5-dihydroimidazol-2-ylidene[2-(i-propoxy)-5-(N,N-dimethylaminosulfonyl)phenyl]methyleneruthenium(II) dichloride (resin supported), Dichloro[1,3-bis(2,4,6-trimethylphenyl)-2-imidazolidinylidene](3-phenyl-1H-inden-1-ylidene)(tricyclohexylphosphine)ruthenium(II), [1,3-Bis(2,4,6-trimethylphenyl)-2-imidazolidinylidene]-[2-[[(4-methylphenyl)imino]methyl]-4-nitrophenolyl]-[3-phenyl-1H-inden-1-ylidene]ruthenium(II) chloride, Dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II), Dichloro[1-(2,6-diisopropylphenyl)-2,2,4-trimethyl-4-phenyl-5-pyrrolidinylidene](2-isopropoxyphenylmethylene)ruthenium(II), [1,3-Bis(2,6-diisopropylphenyl)-2-imidazolidinylidene]dichloro[(2-isopropoxy)(5-trifluoroacetamido)benzylidene]ruthenium(II), Dichloro[1,3-bis(2,6-diisopropylphenyl)-2-imidazolidinylidene](benzylidene)(tricyclohexylphosphine)ruthenium(II), Dichloro[1-(2,4,6trimethylphenyl)-2,2,4-trimethyl-4-phenyl-5-pyrrolidinylidene](2-isopropoxyphenylmethylene)ruthenium(II), Dichloro[1-(2,6-diisopropylphenyl)-2,2,4-trimethyl-4-phenyl-5-pyrrolidinylidene](2-isopropoxyphenylmethylene)ruthenium(II), Dichloro[1,3-bis(2,6-diisopropylphenyl)-2-imidazolidinylidene][(2-isopropoxy)(5-pentafluorobenzoylamino)benzylidene]ruthenium(II), Dichloro[1,3-bis(2,4,6-trimethylphenyl)-2-imidazolidinylidene]{[5-(2-ethoxy-2-oxoethanamido)]-2-isopropoxybenzylidene}ruthenium(II), Dichloro[1,3-bis(2,4,6-trimethylphenyl)-2-imidazolidinylidene][(2-isopropoxy)(5-pentafluorobenzoylamino)benzylidene]ruthenium(II), (1,3-Bis(2,6-diisopropylphenyl)imidazolidin-2-ylidene)dichloro(2-((1-(methoxy(methyl)amino)-3-methyl-1-oxobutan-2-yl)oxy)benzylidene)ruthenium(II), Dichloro[1,3-bis(2,4,6-trimethylphenyl)-2-imidazolidinylidene](3-phenyl-1H-inden-1-ylidene)(pyridyl)ruthenium(II), Dichloro[1,3-bis(2,4,6-trimethylphenyl)-2-imidazolidinylidene][(2-isopropoxy)(5-trifluoroacetamido)benzylidene]ruthenium(II), Dichloro[1,3-bis(2,6-diisopropylphenyl)imidazolidin-2-ylidene][(5-isobutoxycarbonylamino)-(2-isopropoxy)benzylidene]ruthenium(II) and Dichloro[1,3-bis(2,6-diisopropylphenyl)-2-imidazolidinylidene](3-phenyl-1H-inden-1-ylidene)(triphenylphosphine)ruthenium(II).

12. The process according to claim 1, wherein the process further comprises the steps of converting compound of formula (I) to compound of formula (V); 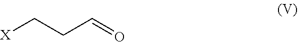
in the form of any one of its stereoisomers or a mixture thereof, and wherein X is a group of formula a) or b) having the same meaning as defined in claim 1.

13. The process according to claim 12, wherein the preparation of compound of formula (V) comprises, when m is 1, a deprotection and hydrogenation step.

14. The process according to claim 12, wherein the preparation of compound of formula (V) comprises, when m is 0, an isomerization and a deprotection step.

15. A compound of formula (VIII): 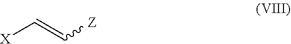
in the form of any one of its stereoisomers or a mixture thereof, and wherein X is a group of formula b) or d) 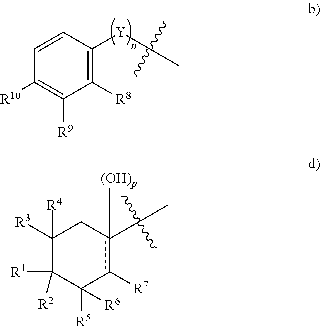
in the form of any one of its stereoisomers, and
wherein p is 0 when the dotted line is a carbon-carbon double bond and p is 1 when the dotted line is a carbon-carbon single bond;
each R¹, R², R³, R⁴, R⁵, R⁶ and R⁷, independently from each other, represent a hydrogen atom, a C₁₋₆ alkyl group or a C₂₋₆ alkenyl group, each optionally substituted by a hydroxy or C₁₋₃ alkoxy group; or two groups among R¹, R², R³, R⁴, R⁵, R⁶ and R⁷ are taken together and form C₃₋₈ cycloalkyl or C₅₋₈ cycloalkenyl group, each optionally substituted by a hydroxy, C₁₋₃ alkyl or C₁₋₃ alkoxy group, and the others groups have the same meaning as defined above;
each R⁸, R⁹ and R¹⁰, independently from each other, represent a hydrogen atom, a C₁₋₆ alkyl group or a C₂₋₆ alkenyl group, each optionally substituted by a hydroxy or C₁₋₃ alkoxy group;
or two groups among R⁸, R⁹ and R¹⁰ are taken together and form C₃₋₈ cycloalkyl or C₅₋₈ cycloalkenyl group, each optionally substituted by a hydroxy, C₁₋₃ alkyl or C₁₋₃ alkoxy group, and the other group has the same meaning as defined above;
n is 0 or 1;
Y is a —CH═CR¹¹—, —CHR¹²—CHR¹¹— or —CH₂—C(OH)R¹¹— group wherein R¹¹ is a hydrogen atom or a methyl or ethyl group and R¹² is a hydroxy or a acetate group;
Z is, when X is of formula d), a CHO group, a CH₂OH group or a CH(OR^{a})(OR^{b})ₘ group wherein m is 0 or 1, R^{a} is a C₁₋₄ alkyl group, a trimethyl silyl group, a C(═O)—R^{c} group, a C(═O)—OR^{c} group, a CH₂(OR^{c}) group, a CH(OR^{c})CH₃ group wherein R^{c} is a C₁₋₄ alkyl group; R^{b} is a C₁₋₄ alkyl group, a C(═O)—R^{c} group wherein R^{c} is a C₁₋₄ alkyl group; or R^{a} and R^{b} are taken together and represent a C₂₋₆ alkanediyl group; or
Z is, when X is of formula b), a CH(OR^{a})(OR^{b}) group wherein R^{a} and R^{b}, independently from each other, are a methyl group or a C₃₋₄ alkyl group, a C(═O)—R^{c} group wherein R^{c} is a C₁₋₄ alkyl group; or R^{a} and R^{b} are taken together and represent a C₃₋₆ alkanediyl group; and
provided that when X is a group of formula (d), then at least one group among R¹, R², R³, R⁴, R⁵, R⁶ and R⁷ is a C₁₋₆ alkyl group or a C₂₋₆ alkenyl group, each optionally substituted by a hydroxy or C₁₋₃ alkoxy group; provided that when X is a group of formula (d) and six groups among R¹, R², R³, R⁴, R⁵, R⁶ and R⁷ are a hydrogen atom, then the group among R¹, R², R³, R⁴, R⁵, R⁶ and R⁷ being not a hydrogen atom is not a methyl group; provided that when X is a group of formula (d) and R², R³, R⁴, R⁵, R⁶ and R⁷ are a hydrogen atom then R¹ is not a prop-1-en-2-yl group; and provided that 3-(4-(tert-butyl)cyclohex-1-en-1-yl)acrylaldehyde, 3-(4-(tert-butyl)-1-hydroxycyclohexyl)acrylaldehyde, 1-(tert-butyl)-4-(3,3-diethoxyprop-1-en-1-yl)benzene, 2-(4-(tert-butyl)styryl)-1,3-dioxolane, 3-(4-(tert-butyl)-1-hydroxycyclohexyl)allyl acetate, 3-(2-allyl-1-hydroxycyclohexyl)allyl acetate, 4-(tert-butyl)-1-(3-hydroxyprop-1-en-1-yl)cyclohexan-1-ol, 2-allyl-1-(3-hydroxyprop-1-en-1-yl)cyclohexan-1-ol, 2-(4-isopropylstyryl)-1,3-dioxane, 2-(4-isopropylstyryl)-4-methyl-1,3-dioxolane, 3-(2-(buta-1,3-dien-1-yl)-1-hydroxycyclohexyl)acrylaldehyde, 3-(5,5-dimethylcyclohex-1-en-1-yl)acrylaldehyde, 3-((1S,5R)-6,6-dimethylbicyclo[3.1.1]hept-2-en-3-yl)acrylaldehyde, (1R,2S,5R)-1-(3-(tert-butoxy)prop-1-en-1-yl)-2-isopropyl-5-methylcyclohexan-1-ol, 2-allyl-1-(3-hydroxyprop-1-en-1-yl)cyclohexan-1-ol, (3,3-dimethoxyprop-1-en-1-yl)benzene, are excluded.
